(19) United States
(12) Patent Application Publication
(10) Pub. No.: US 2025/0262410 A1
(43) Pub. Date: Aug. 21, 2025

(54) MAGNETICALLY STEERABLE CATHETER

(71) Applicant: BOARD OF REGENTS, THE UNIVERSITY OF TEXAS SYSTEM, Austin, TX (US)

(72) Inventors: Farshid ALAMBEIGI, Austin, TX (US); Lei ZHOU, Austin, TX (US); Mohammad Reza RAJEBI, Corpus Christi, TX (US)

(21) Appl. No.: 18/857,661

(22) PCT Filed: Apr. 18, 2023
(86) PCT No.: PCT/US2023/065872

§ 371 Date: Oct. 17, 2024

Related U.S. Application Data
(60) Provisional application No. 63/332,121, filed on Apr. 18, 2022.

(51) Int. Cl. A61M 25/01; A61M 25/00

(52) U.S. Cl. CPC A61M 25/0127; A61M 25/0013; A61M 25/005; A61M 25/0158; A61M 2025/0166; A61M 2205/70

(57) ABSTRACT

Exemplary embodiments of the present disclosure apparatus and methods for enhancing the steerability of catheters, including for example those used in peripheral artery disease PAD interventions. Exemplary embodiments include a permanent magnet coupled to an end of a segmented tubular member, an electromagnet, and a controller configured to control a magnetic force exerted by the electromagnet on the permanent magnet. Exemplary embodiments further comprise systems and methods for detecting the location of a magnetic element via magnetic sensors.

### CROSS-REFERENCE TO RELATED APPLICATIONS

This application claims priority to U.S. Provisional Patent Application Ser. No. 63/332,121 filed Apr. 18, 2022, the entire contents of which are incorporated by reference herein.

### BACKGROUND INFORMATION

Peripheral artery disease (PAD) is a medical condition where the patient's leg blood vessels are obstructed or narrowed, which is typically treated by a percutaneous endovascular intervention (PEI). In a PEI, a catheter is inserted and steered into the affected blood vessel with the goal of opening the blocked artery and restoring the blood flow. Existing manual PEI procedures suffer from low steerability of catheters, which incur challenges to navigate the catheter through complex vasculatures with bifurcations, causing increased radiation exposure and trauma risks in the tissue. Magnetic actuation is a promising approach for non-contact catheter steering. However, existing designs of magnetically steerable continuum robotic catheters often suffer from limited steering capability, high cost, and the lack of intuitive user interface, making them challenging to be deployed clinically.

More than 200 million individuals worldwide have been afflicted with peripheral artery disease (PAD) by 2010 [1], [2]. Under such a medical condition, the patient's leg blood vessels are obstructed or narrowed, affecting the normal blood flow through the body, which causes pain, impaired walking, intermittent claudication, and critical limb ischemia in the involved patients [3]. PEI is the primary treatment strategy for patients with PAD to properly restore the blood flow through the involved arteries [4]. In this minimally invasive procedure, a radiologist first uses ultrasound or fluoroscopic imaging to locate the blockage or narrowed part of the blood vessel. Through a small incision, he or she then inserts a catheter into the affected blood vessel and carefully steers it to the blocked area of interest under the fluoroscopic imaging guidance. Next, a balloon is deployed and inflated through the catheter to open the blocked vessel to restore the blood flow. If necessary, a stent is also passed through the catheter and implanted to keep the vessel permanently open.

Despite the benefits of current PEIs, these minimally invasive manual procedures often suffer from low steerability of the existing catheters, making the PEI procedure very demanding, arduous, and time-consuming [4]. This limitation is more evident while steering a flexible catheter through complex leg vasculatures with small and variable diameters, vessel bifurcations, and non-uniform branches. Furthermore, aside from lengthy PEIs, increased X-ray radiation exposure to both patients and clinicians is another side-effect of utilizing existing catheters. The mentioned shortcomings can be attributed to these medical instrument's mechanical design and fabrication procedure and the relatively steep learning curve associated with their usage. Of note, the latter makes the utilization of these devices often unintuitive and heavily based on the operator's experience and skills [4], [5].

Steerable catheters is an effective approach to address these limitations, which is typically driven via tendons or guidewires [6]. As an alternative approach, the magnetically actuated steerable catheters can be made at smaller size and with simpler structure, and has recently attracted considerable interests to treat a wide range of PEIs. For instance, Jeon et al. developed a magnetically actuated flexible guidewire robotic system for performing 2D [7] and 3D [8] coronary interventions in blood vessel phantoms using a commercially-available eight-coil electromagnetic system. In addition, Kim et al. [9] developed a magnetic field-controllable elastomeric fiber that has been used as a ferromagnetic soft robotic guidewire to improve three-dimensional navigation in cerebrovascular interventions. To remotely steer the guidewire in a three-dimensional printed phantom, authors have used a relatively large cylindrical permanent magnet to manually manipulate the robot in a desired direction. In a recent study, Wang et al. [10], proposed a model-based design and fabrication procedure to improve the workspace and performance of this type of soft guidewires for cardiovascular diseases.

Aside from robotic guidewires, literature also documents different types of magnetically actuated robotic catheters that have mainly been developed for cardiac ablation PEIs [11]. For instance, Losey et al. developed a magnetically actuated remote-controlled catheter with a solenoid coil at its distal tip. The performed experiments under an MRI system clearly demonstrated the catheter navigation capability of the proposed system which outperforms that of manual PEI. In a more recent study, a nitinol-made monolithic continuum manipulator with flexure patterns along its body has been presented targeting various PEIs [13]. To create a larger deflection force and rapidly change the orientation of the outer magnets, a three-magnet-tip catheter design have also been proposed by various groups (e.g., [14]). Moreover, variable stiffness magnetically steerable catheters using low melting point alloys have also been introduced recently to improve the steerability of single-magnet robots in complex vascular anatomies [15]. Overall, review of the literature demonstrates that most of the developed guidewire and catheter robotic systems use a solid permanent magnet (PM) at their distal tip (e.g., [15]) that blocks the feasibility of passing an instrument or stent through their internal channel, which is crucial for various PEIs such as PAD treatment. Also, most prior-developed magnetically steerable robotic catheters rely on manually actuation using external permanent magnets (e.g., [16]) or bulky external magnetic actuators such as eight-coil electromagnetic system to provide adequate electromagnetic field for actuation of these robots. Nevertheless, similar to the PAD treatment, various PEIs demand a more compact electromagnetic actuation system that do not disturb the conventional surgical workflow, while minimizing the X-ray radiation exposure and providing a more intuitive steering capability for the clinicians.

Accordingly, magnetically steerable robotic catheters (MSRC) are a promising technology for percutaneous endovascular intervention (PEI) procedures to treat peripheral arterial diseases (PAD), where magnetic actuation is used to steer the catheter tip during navigation. However, current MSRC systems require fluoroscopic imaging for catheter location sensing during the procedure, which risks creating radiation-induced injuries to both the patient and the radiologist.

Accordingly, systems and methods are desired that overcome these and other limitations associated with existing systems and methods.

### SUMMARY

Exemplary embodiments of the present disclosure include apparatus and methods for enhancing the steerability of catheters used in PAD interventions and overcoming the aforementioned limitations of current systems. Exemplary embodiments include a magnetically steerable robotic catheter with a ring-shaped magnet and hollow instrument channel. Exemplary embodiments offer an easy-to-fabricate and cost-effective procedure for creating kirigami-type flexure patterns on the distal end of the robotic catheter for flexibility variation. The added flexibility caused by such features mitigates the need for having bulky external electromagnetic actuation systems while maximizing the steerability of the catheter in complex vasculature and bifurcations. Furthermore, to minimize the X-ray radiation exposure and improve the intuitiveness of the demanding PEI procedure, exemplary embodiments include a remote control user-interface to navigate the developed two degree-of-freedom (DoF) catheter within the vasculature. The performance of the proposed robotic catheter system has been experimentally validated through fabricated phantoms emulating the bifurcations in the leg vasculature.

Certain embodiments include a cost effective fabrication approach for fabricating a flexure-based distal segment using the heat-shrinking property of thermoplastic materials. Contactless magnetic actuation based on a ring-shape magnet can also be utilized for the steering of the catheter distal segment together with a compact friction drive for insertion automation.

As discussed herein, the obtained Finite Element simulation and experimental results had high correlation and clearly validated the efficacy and high performance of the fabrication procedure and magnetically steerable robotic system. Moreover, experiments of remotely navigating the catheter along a custom-designed leg vasculature phantom indicated the system's steerability and maneuverability.

Aiming to reduce the duration of x-ray radiation in interventions using MSRCs, exemplary embodiments of the present disclosure include a steerable robotic catheter system that integrates magnetic location sensing and magnetic actuation.

Specific embodiments include a catheter with a magnetic tip to enable steering by external electromagnetic actuators. In addition, a cylindrical array of magnetic sensors is used to measure the field from the catheter tip to enable real-time catheter location estimation. To enable improved localization accuracy, a novel nested calibration algorithm for sensor positions and magnet dipole strength is introduced. Exemplary embodiments further include a novel integration method for magnetic actuation and magnetic location sensing in MSRC systems, where fluoroscopic imaging is only required during catheter steering at bifurcations in the vasculatures, which effectively reduces x-ray radiation in the navigation process of catheters.

The proposed methodology is tested with an MSRC prototype, where a ring-shaped magnet (3 mm OD, 1.6 mm ID, 6 mm length) is arranged at the catheter tip for actuation and location sensing. The magnet location estimation algorithm is implemented for real-time visual feedback to the operator with a low latency of 400 ms. Experiments show that an average position estimation error of 1.27 mm can be achieved after calibrating the system using the proposed algorithm. The prototype catheter can successfully navigate through phantoms of vasculatures under a combination of magnetic steering and magnetic location sensing, which shows the excellent potential of the proposed framework for reducing x-ray exposure in PAD interventions.

Certain embodiments include an apparatus comprising a permanent magnet, a segmented tubular member comprising a first end and a second end, an electromagnet, and a controller, where: the permanent magnet is coupled to the first end of the segmented tubular member; the second end of the segmented tubular member is configured to receive a distal end of a catheter; the controller is configured to control an electrical current to the electromagnet, and the controller is configured to control a magnetic force exerted by the electromagnet on the permanent magnet.

In particular embodiments, the permanent magnet is a first permanent magnet, and the apparatus further comprises a second permanent magnet coupled to the segmented tubular member between the first end of the segmented tubular member and the second end of the segmented tubular member. Some embodiments further comprise a third permanent magnet coupled to the segmented tubular member between the first end of the segmented tubular member and the second end of the segmented tubular member. Specific embodiments further comprise a drive mechanism configured to engage a catheter inserted into the first end of the segmented tubular member. Certain embodiments further comprise a catheter inserted into the first end of the segmented tubular member. In particular embodiments the permanent magnet comprises a central channel, and in some embodiments the electromagnet comprises a pair of Helmholtz coils.

In specific embodiments the pair of Helmholtz coils is a first pair of Helmholtz coils; the electromagnet comprises a second pair of Helmholtz coils; and the controller is configured to control a magnetic force exerted by the electromagnet on the permanent magnet in three dimensional space. In certain embodiments the electromagnet comprises a third pair of Helmholtz coils, and particular embodiments further comprise an imaging device. In some embodiments the imaging device is configured to provide imaging data of the permanent magnet. In specific embodiments the permanent magnet and the segmented tubular member each have an outer diameter between 0.1 millimeters and 10 millimeters. In certain embodiments the permanent magnet and the segmented tubular member each have an outer diameter less than or equal to 6.0 millimeters or more specifically the permanent magnet and the segmented tubular member each have an outer diameter less than or equal to 4.0 millimeters. In particular embodiments the segmented tubular member comprises a rectangular pattern of voids.

Specific embodiments include a magnetic sensor configured to detect a position of the permanent magnet. In certain embodiments the magnetic sensor is configured as an array of sensors circumscribing the permanent magnet. In some embodiments the sensor is coupled to the controller, and specific embodiments further comprise a visual display configured to display a position of the permanent magnet.

Specific embodiments include a method of directing a catheter into a lumen, the method comprising: inserting the catheter into a segmented tubular member (where the segmented tubular member comprises a first end and a second end; the segmented tubular member comprises a permanent magnet coupled to the first end; and the catheter is inserted into the second end of the segmented tubular member); advancing the catheter and the segmented tubular member into the lumen; and exerting a magnetic force on the permanent magnet.

In certain embodiments the lumen is an artery with an inner diameter greater than or equal to 6.0 millimeters. In particular embodiments the magnetic force is generated by an electromagnet. Some embodiments further comprise varying the magnetic force generated by the electromagnet by controlling an electrical current to the electromagnet. In specific embodiments the electromagnet comprises a pair of Helmholtz coils. In certain embodiments advancing the catheter and the segmented tubular member into the lumen comprises engaging the catheter with a drive mechanism. In particular embodiments the permanent magnet comprises a central channel, and the method further comprises inserting an instrument through the central channel.

Some embodiments further comprise obtaining imaging data of the permanent magnet. In particular embodiments the permanent magnet and the segmented tubular member each have an outer diameter less than or equal to 6.0 millimeters. In some embodiments the permanent magnet and the segmented tubular member each have an outer diameter less than or equal to 4.0 millimeters. In specific embodiments the segmented tubular member comprises a rectangular pattern of voids.

Certain embodiments include a method of making a segmented tubular member, the method comprising: obtaining a flexible tubular member; obtaining a pattern; flattening the flexible tubular member; positioning the pattern on the flexible tubular member; using a cutting instrument to remove material from flexible tubular member, wherein the cutting instrument follows the pattern; and removing the pattern from the flexible tubular member.

Particular embodiments include a method of directing a magnetic element into a lumen, where the method comprises: obtaining a radiographic image of the lumen, where the lumen comprises a non-bifurcated portion and a bifurcated portion, and where the bifurcated portion of the lumen comprises a target branch and a non-target branch; inserting the magnetic element into the non-bifurcated portion of the lumen, where a position of the magnetic element is tracked via a magnetic sensor until the magnetic element is proximal to the bifurcated portion; imaging the magnetic element via radiography; directing the magnetic element into the target branch of the bifurcated portion of the lumen via a magnetic force on the permanent magnet; advancing the magnetic element into the target branch of the bifurcated portion of the lumen; ceasing imaging of the magnetic element via radiography; and detecting the position of the magnetic element via a magnetic sensor.

In specific embodiments the magnetic element is coupled to a distal end of a catheter. In certain embodiments the lumen is an artery. In particular embodiments imaging the magnetic element via radiography comprises x-ray imaging. In some embodiments the magnetic force on the permanent magnet is generated by an electromagnet. In specific embodiments the magnetic force on the permanent magnet is generated by a pair of Helmholtz coils. In certain embodiments advancing the magnetic element into the target branch of the bifurcated portion of the lumen comprises engaging the catheter with a drive mechanism. In particular embodiments the permanent magnet comprises a central channel, and the method further comprises inserting an instrument through the central channel. In some embodiments the permanent magnet has an outer diameter less than or equal to 6.0 millimeters. In specific embodiments the permanent magnet has an outer diameter less than or equal to 4.0 millimeters. Certain embodiments comprise inserting the distal end of the catheter into a segmented tubular member prior to inserting the magnetic element into the non-bifurcated portion of the lumen. In particular embodiments the segmented tubular member comprises a rectangular pattern of voids.

Certain embodiments include an apparatus comprising: a permanent magnet; an electromagnet; a magnetic sensor configured to detect a position of the permanent magnet; and a controller, where the controller is configured to control an electrical current to the electromagnet, and to control a magnetic force exerted by the electromagnet on the permanent magnet. Particular embodiments further comprise a segmented tubular member, where the segmented tubular member comprises a first end and a second end. and the permanent magnet is coupled to the first end of the segmented tubular member. In some embodiments the second end of the segmented tubular member is configured to receive a distal end of a catheter. In specific embodiments the electromagnet comprises a pair of Helmholtz coils. In certain embodiments the pair of Helmholtz coils is a first pair of Helmholtz coils; the electromagnet comprises a second pair of Helmholtz coils; and the controller is configured to control a magnetic force exerted by the electromagnet on the permanent magnet in three-dimensional space. In particular embodiments the electromagnet comprises a third pair of Helmholtz coils. Some embodiments further comprise an imaging device.

In specific embodiments the imaging device is configured to provide imaging data of the permanent magnet. In certain embodiments the permanent magnet and the segmented tubular member each have an outer diameter between 0.1 millimeters and 10 millimeters. In some embodiments the permanent magnet and the segmented tubular member each have an outer diameter less than or equal to 6.0 millimeters. In particular embodiments the permanent magnet and the segmented tubular member each have an outer diameter less than or equal to 4.0 millimeters. In specific embodiments the segmented tubular member comprises a rectangular pattern of voids.

Certain embodiments include a method of detecting a position of a magnetic element, the method comprising: (a) placing the magnetic element in a plurality of known positions; (b) estimating a position of the magnetic element by measuring a magnetic field of the magnetic element in the plurality of known positions via a plurality of magnetic sensors; and (c) calculating a position of the magnetic element, where: each magnetic sensor in the plurality of magnetic sensors is located in a fixed position; and calculating the position of the magnetic element is performed via a nested least square method. In particular embodiments the nested least square method minimizes discrepancies between the known position of the magnetic element and the estimated position of the magnetic element using the measured magnetic field and the fixed position of the sensor.

In the following, the term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

As used herein, the terms “cut” (and related terms such as “cutting”, etc.) and “break the mechanical integrity” (and related phrases such as “breaking the mechanical integrity”) are used to refer to a process of breaking the molecular bonds in tissue.

As used herein, the term “light source” is understood to include any source of electromagnetic radiation, including for example, a laser. It is also understood that a “first light source” and a “second light source” may originate from a single laser. For example, a laser configured for operating under a first set of parameters (e.g. wavelength, amplitude, continuous wave or continuous pulse mode) may be considered a “first light source”, while the same laser configured for operating under a second set of parameters may be considered a “second light source.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will be apparent to those skilled in the art from this detailed description.

### BRIEF DESCRIPTION OF THE DRAWINGS

The following drawings form part of the present specification and are included to further demonstrate certain aspects of the present disclosure. The invention may be better understood by reference to one of these drawings in combination with the detailed description of specific embodiments presented herein.

FIG. 1 shows a schematic view of an apparatus according to an exemplary embodiment of the present disclosure during use.

FIG. 2 shows steps in one procedure for fabricating a segmented tubular member according to the embodiment shown in FIG. 1.

FIG. 3 shows a schematic view of the magnetic forces exerted upon a permanent magnet by an electromagnet according to the embodiment shown in FIG. 1.

FIG. 4 shows data of a model validation for magnetic steering torque generation and the variation of simulated steering torque with respect to the current the electromagnet according to the embodiment shown in FIG. 1.

FIG. 5 shows a schematic view of a drive mechanism according to the embodiment shown in FIG. 1.

FIG. 6 shows a mechanical finite element simulation for the segmented tubular member according to the embodiment shown in FIG. 1.

FIG. 7 shows a catheter steering and insertion experimental setup according to the embodiment shown in FIG. 1.

FIG. 8 shows simulated and experimental data for displacement variation with respect to the current amplitude according to the embodiment shown in FIG. 1.

FIG. 9 illustrates the photographs of an apparatus according to the embodiment shown in FIG. 1 during use.

FIG. 10 illustrates a schematic of a robotic system including the embodiment shown in FIG. 1 during use.

FIG. 11 illustrates a conceptual illustration of a magnetically steerable robotic catheters (MSRC) with planar deformation and flexure patterns actuated by a magnetic field generated by the Helmholtz coils. The zoomed region A-A shows the cross sections of the tubular section and zoomed region B-B shows the cross sections of the notched sections.

FIG. 12 illustrates simulation results of MSRCs with S1 design actuated by an input current ranging from 0 to approximately 40 amperes.

FIG. 13 illustrates simulation results of MSRCs with five different notch geometries actuated with an identical input current I=40 N.

FIG. 14 illustrates simulation results of an MSRC experiencing four external loading scenarios L1*˜L4* and are actuated by input current I=40 N.

FIG. 15 illustrates a comparison of the tip displacements of an embodiment actuated with input current ranging from 0 to approximately 13 amperes, and the tip displacement obtained from the proposed model.

FIG. 16 illustrates a side view of an apparatus comprising magnetic sensors according to an exemplary embodiment of the present disclosure during use.

FIG. 17 illustrates an isometric view of the embodiment of FIG. 15 during use.

FIG. 18 illustrates a schematic diagram showing the variables in magnetic localization according to an exemplary embodiment of the present disclosure.

FIG. 19 illustrates a nested calibration method for magnetic dipole strength and sensor position according to an exemplary embodiment of the present disclosure.

FIG. 20 illustrates views of navigating a robotic catheter through a bifurcation in the vascular structure under a combination of magnetic steering, magnetic localization, and x-ray fluoroscopic imaging according to an exemplary embodiment of the present disclosure.

FIG. 21 illustrates a catheter steering and insertion with magnetic localization experimental setup according to the embodiment shown in FIG. 16.

FIG. 22 illustrates magnetic localization data without sensor location calibration.

FIG. 23 illustrates calibration problem objective value data under varying dipole moment M according to an exemplary embodiment.

FIG. 24 illustrates magnet location estimation data using an angled calibration plate after the sensor position and magnetic dipole strength calibrated using a nested algorithm as disclosed herein, with data in FIG. 22 used for calibration.

FIG. 25 illustrates a comparison in magnet volume/sensor-to-magnet distance [mm³/mm] and tracking accuracy between exemplary embodiments of the present disclosure and prior studies.

FIG. 26 illustrates photographic images of digital microscope video for an exemplary embodiment of the present disclosure navigating through a 3D-printed vascular phantom and the screenshots of the user interface showing the catheter tip location estimation using magnetic localization.

FIG. 27 illustrates catheter localization steering data from Y-shaped and S-shaped phantoms testing as user inserted and retracted magnet.

### DETAILED DESCRIPTION OF ILLUSTRATIVE EMBODIMENTS

Exemplary embodiments of the present disclosure include apparatus and methods for treatment of peripheral artery disease (PAD), including for example, the use of magnetically steerable catheters Exemplary embodiments also include apparatus and methods for detecting the location of magnetic elements (including, but not limited to, magnetically steerable catheters) via magnetic sensors.

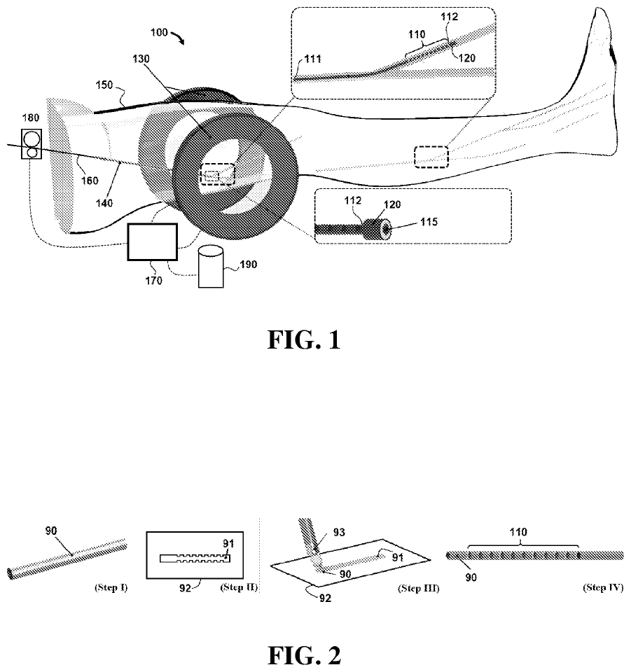

Referring originally to FIG. 1, an apparatus 100 comprises a segmented tubular member 110 with a first end 111, a second end 112 and a central channel 115. First end 111 is configured to receive a catheter 160, and apparatus 100 further comprises a permanent magnet 120 coupled to second end 112 of segmented tubular member 110. While the embodiment shown illustrates a single permanent magnet 120 coupled to second end 112, it is understood that other embodiments may comprise one or more additional permanent magnets coupled to segmented tubular member 110 in additional locations along segmented tubular member 110. In the illustrated embodiment, apparatus 100 also comprises an electromagnet 130, which may be configured as a pair of Helmholtz coils in particular embodiments. In FIG. 1, segmented tubular member 110 and catheter 160 has been inserted into an artery 140 (e.g. a popliteal artery) of a patient 150. In exemplary embodiments, segmented tubular member 110 and permanent magnet 120 can be inserted into an artery with an inside diameter of approximately 5.0 millimeters or larger.

Apparatus 100 also comprises a controller 170 configured to control an electrical current to electromagnet 130 and control a magnetic force exerted by electromagnet 130 on permanent magnet 120. During use of apparatus 100, controller 170 can be operated to control the position of permanent magnet 120 with respect to electromagnet 130. Accordingly, controller 170 can be used to guide permanent magnet 120 as it is advanced through artery 140 and into smaller diameter lumens in the vascular network, including for example arteries with an inside diameter of approximately 3.0 millimeters or larger. In particular embodiments, permanent magnet 120 and segmented tubular member 110 may each have an outer diameter between 0.1 millimeters and 10 millimeters. Segmented tubular member 110 is coupled to permanent magnet 120, and is therefore also guided by controller 170. As discussed in further detail below, catheter 160 may be advanced into or retracted from artery 140 via a drive mechanism 180, which may also be controlled via controller 170. Accordingly, segmented tubular member 110 can be directed to a desired location in an artery or other lumen in patient 150 by controller 170. In certain embodiments, apparatus 100 may also comprise an imaging device 190 to provide imaging data (e.g. the location with patient 150) of permanent magnet 120 and distal second end 112 of segmented tubular member 110.

Segmented tubular member 100 provides flexibility that allows permanent magnet 120 to be manipulated such that second end 112 can be directed at significant angles (e.g. approximately 30 degrees in certain embodiments) and lateral distances (e.g. approximately 12 millimeters in certain embodiments). In embodiments comprising multiple permanent magnets, segmented tubular member 110 can be manipulated in multiple directions, including for example, “S-shaped” configurations. FIG. 2 illustrates steps in one procedure for fabricating segmented tubular member 100. It is understood that the steps shown in FIG. 2 are merely exemplary of one fabrication procedure, and other embodiments may incorporate different steps than those shown. In FIG. 2 Step I, a tubular member 90 is obtained. In certain embodiments, tubular member 90 may be formed from a flexible polymer material, including for example, heat-shrink tubing. In Step II of FIG. 2, a desired pattern 91 is formed on a sheet 92 that may be transparent or translucent in certain embodiments. As shown in Step III, sheet 92 is laid on top of tubular member 90, which is flattened and positioned such that pattern 91 is located on tubular member 90. A cutting instrument 93 can then be used to follow pattern 91 and remove material (e.g. cut) flattened tubular member 90. In certain embodiments, cutting instrument 93 may be a knife, a laser, or other suitable instrument capable of removing material from tubular member 90. In step IV, sheet 92 has been removed from tubular member 90, which is no longer flattened and has returned to its tubular or cylindrical shape. As shown in step IV, segmented tubular member 110 is formed by creating voids in the material removed from tubular member 90. Segmented tubular member 110 therefore allows increased flexibility due to the decreased stiffness resulting from the material removal. While a rectangular pattern of voids is shown in FIG. 2, it is understood that different patterns, including for example, semi-circles, triangles, or other shapes, can be used as a pattern to form the segmented tubular member in other embodiments.

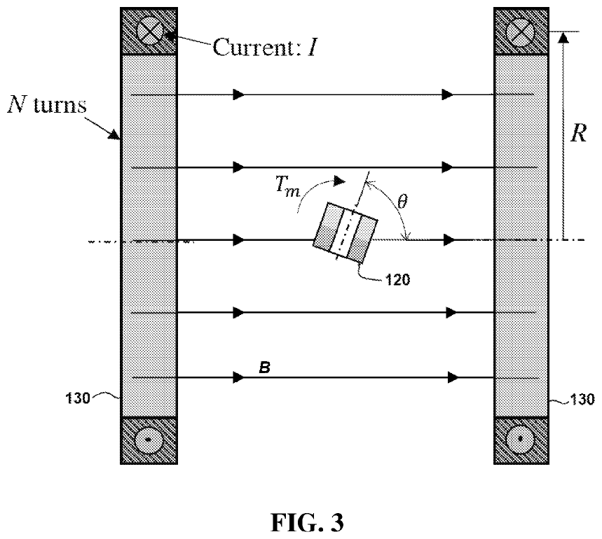

FIG. 3 provides a schematic view of the magnetic forces exerted upon permanent magnet 120 by electromagnet 130. In this embodiment, permanent magnet 120 is a ring-shaped magnet and electromagnet 130 is configured as a pair of Helmholtz coils—i.e., a pair of coils with distance equal to their radius [21] are used to generate approximately uniform magnetic field between the coils. Unlike other magnetically steerable catheters in the literature (e.g., [18]), a single ring-shape axially-magnetized permanent magnet 120 is arranged at the catheter distal segment to provide an instrument channel and avoid blocking the distal tip of the proposed catheter. When currents are excited in the coils, steering torque is generated to the permanent magnet 120, which controls the direction of the catheter's distal segment. Note that although only planar bending is presented in illustrated embodiments, such actuation principle can be readily extended to three dimensional steering actuation.

The magnetic flux density generated by the Helmholtz coils in electromagnet 130 can be obtained as [22]

$$B = \frac{8}{5 \sqrt{5}} \frac{{μ}_{0} NI}{{R}_{c}} , \qquad ( 1 )$$

where μ₀=4π10⁻⁷ T·m/A is the permeability of free space, N is the number of turns in each coil, I is the current amplitude in coils, and R is the coil radius.

Due to the small size of the utilized magnet, magnet 120 can be modeled as a magnetic dipole with a dipole moment of

$$M = \frac{{B}_{r} {V}_{m}}{{μ}_{0}} , \qquad ( 2 )$$

where Bᵣ is the remanence of the permanent magnet material and Vₘ is the volume of the magnet [21].

The steering torque generated on the magnet can then be calculated as

$${T}_{m} = MB sin θ , \qquad ( 3 )$$

where θ is the angle between the magnetic dipole momentand the external magnetic field. With (1)-(3), the relationship between current/and steering torque Tₘ can be found.

The magnetic steering torque generated to permanent magnet 120 can be calculated by three-dimensional magnetostatic finite element (FE) simulations using Ansys Maxwell (Ansys, Inc., USA). Note that fine mesh near the magnet is considered to accurately capture the magnetic torque generation.

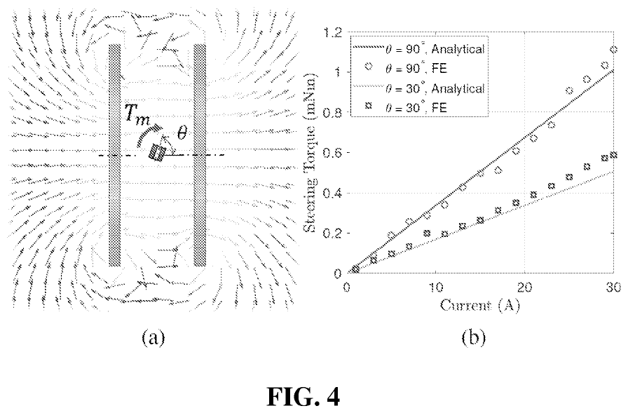

A model validation for magnetic steering torque generation is shown in FIG. 4(a), which illustrates a FE simulated magnetic field generated by Helmholtz coils and magnet diagram using Ansys Maxwell. FIG. 4(b) shows the variation of FE simulated steering torque with respect to the current in coils. As shown in the figure, good agreement between the analytical model and the FE simulation results validates the model.

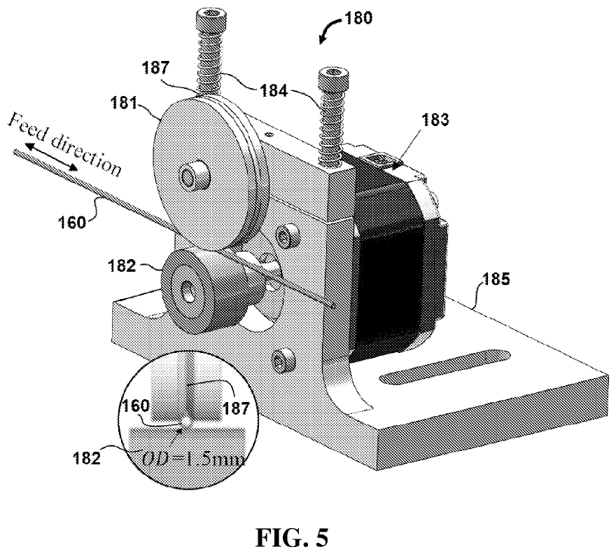

FIG. 5 illustrates one embodiment of drive mechanism 180 configured as a compact catheter feeding mechanism to insert catheter 160. The use of drive mechanism 180 can enable remote operation of PEIs and thereby minimize the radiation exposure to a surgeon through the procedure.

In this embodiment, drive mechanism 180 uses a friction drive [23], [24] which comprises an idler wheel 181 to position and press catheter 160 and a drive wheel 182 for pushing the catheter's feed motion via friction. In certain embodiments, drive wheel 182 can comprise a thick layer of urethane sleeve covering its outer surface to enable effective pushing for a variety of catheters with different dimensions and materials. In particular embodiments, idler wheel 181 comprises a V-shaped groove 187 to constrain catheter 160 in the lateral direction. To minimize wear of the catheter and the driving wheels, idler wheel 181 may be formed from a material comprising a low friction solid polymer, which allows catheter 160 to freely slip with respect to idler wheel 181 without damage. In the illustrated embodiment, a stepper motor 183 is mounted to a bracket 185 and is used to control the motion of drive wheel 182. The pushing force that drive mechanism 180 can apply to catheter 160 is determined by the maximum friction force between the drive wheel 182 and catheter 160, which is dependent in part on the stiffness of preload springs 184.

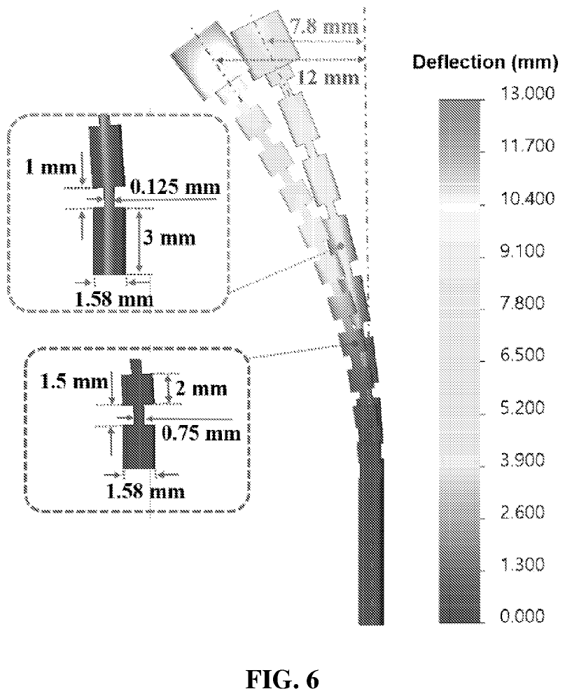

FIG. 6 illustrates a mechanical finite element simulation for one exemplary embodiment of a pattern for a segmented tubular member according to the present disclosure. To effectively design a flexure pattern for segmented tubular member 110 that enables the catheter to remotely steer in the shown leg's bifurcations in FIGS. 1 and 2, FE simulations module in SolidWorks (Dassault Syste'mes®) were used to analyze the bending behavior of the distal segment under the actuation torques generated by the Helmholtz coils. This mechanical FE model considers the calculated steering torque obtained in Ansys Maxwell as the input (shown in FIG. 4), and then calculates the catheter's planar bending displacement and behavior. FIG. 6 illustrates the simulated maximum achievable bending displacement of 7.8 and 2 mm of the catheter distal segment with two different flexure patterns under same current amplitude in the coils. Of note, this simulation was used to optimize and select the desired flexure pattern for the examples discussed below.

### Examples

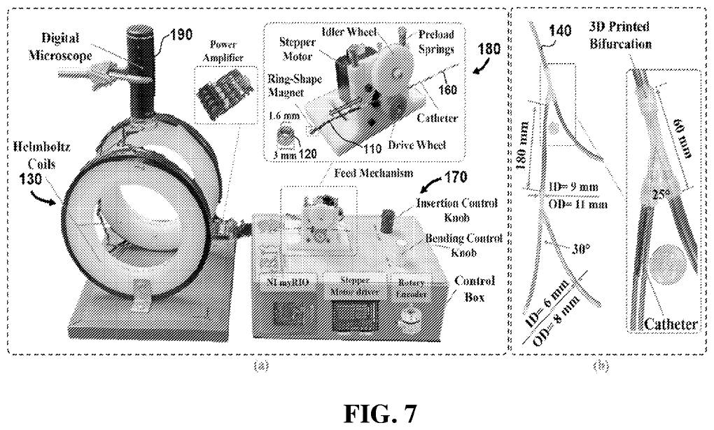

FIG. 7 shows the overview of the catheter steering and insertion experimental setup with specific component descriptions for one exemplary embodiment. For purposes of clarity, not all components in FIG. 7 are labeled with reference numbers. The embodiment shown in FIG. 7 comprises a magnetic steering system with electromagnets 130, catheter 160 with a flexible distal segment 110 and a ring-shape magnet 120 adhered at its distal end, a catheter feeding mechanism comprising drive mechanism 180, a control box for remote operation interface as controller 170, a digital microscope simulating the fluoroscopic images in a real PEI as the visual feedback on the catheter for imaging device 190, and the leg vasculature phantom simulating artery 140. Based on the performed FEsimulations described in Section II-B and shown in FIG. 4, a magnetic steering system that uses a pair of custom-fabricated Helmholtz coils to createa uniform magnetic field for steering torque generation to the tip of the catheter was designed and fabricated. In this embodiment, the Helmholtz coils have a diameter of 220 mm and are configured with a distance of 110 mm. The coils are connected in series and are driven by a full-bridge bi-directional switching-type power amplifiers (B07NP6XNPR, Cytron Technologies©) with 20 A current capacity. The coils have 120 turns each, and under the maximum allowed current amplitude of 13 amperes (limited by the power supply being selected), the coils can generate a steering field of 25 millitesla (mT).

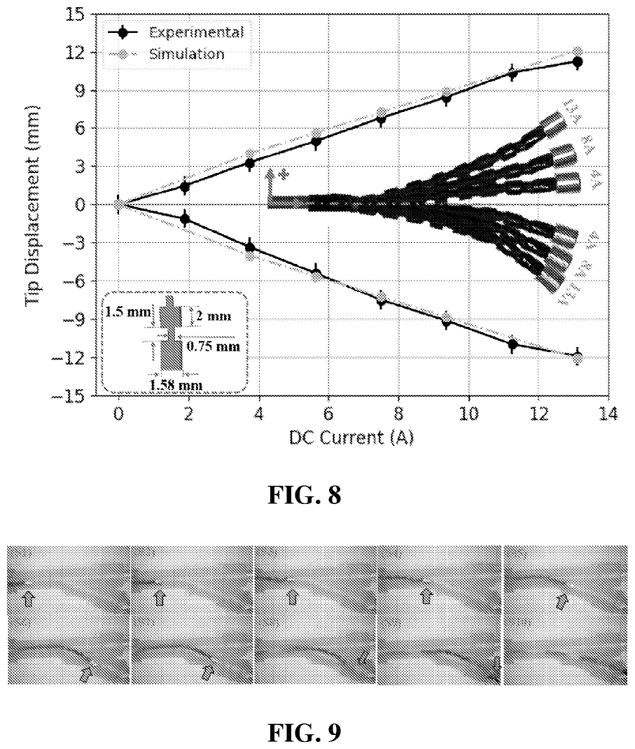

To fabricate the catheter's flexible distal segment, heat shrink tubing (40142000, MILAPEAK) was used with 3.2 mm pre-shrinking outer diameter, 40 mm length, and diagonal shrinking scale of 2:1. Next, based on the performed FE simulations shown in FIG. 6 and the leg's vasculature anatomy (FIG. 7(b)), the flexure pattern was selected generating the 12 mm maximum deflection at the distal end. This configuration provides adequate deflection for the remotely steering of the catheter within the fabricated vasculature phantom. This flexure pattern was printed on an overhead projector film (OHP18-INK-A4x20, Uinkit) and the pattern precisely cut on the heat shrink tubing using a precision swivel knife (SW-600GP, NT Incorporated), following the steps shown in FIG. 2. Finally, heat was applied to the catheter distal segment to obtain the pattern and dimensions shown in FIG. 8. The fabricated flexible segment was then adhered to a commercially available catheter (G11430, Cook Medical, USA) with outer diameter of 1.5 mm from its proximal end. A ring-shaped magnet (N52 grade NdFeB, K&J Magnetics Inc.) with 3 mm outer diameter and 1.6 mm inner diameter was also adhered to the distal end of the flexible segment (FIG. 7(a)).

As shown in FIG. 7(a), the catheter feeding mechanism uses a NEMA 17 stepper motor and implements the friction driven design shown in FIG. 5, where the catheter's insertion motion is driven by the friction force between the drive wheel and the catheter. The pressing/normal force on the catheter was determined by the stiffness of the selected preload springs (i.e., total stiffness of 3.2 Newtons per millimeter [N/mm]). The largest compression of the springs were 5 mm, which exerted a maximum pressing force of 16 N on the catheter. Assuming the static friction coefficient between the catheter and the driving wheel is 0.5, the maximum achievable pushing force on the catheter was calculated as 8 N, which was adequate for the performed experiments.

The remote control interface consists of a box housing two control knobs driving two optical encoders (Taiss® Inc., 600 counts/revolution), a myRIO embedded controller (National Instruments®), and a stepper motor driver (Stepperonline™). One knob is controlling the catheter insertion motion, and the other is controlling the steering of the catheter distal segment. The system is controlled using a real-time controller implemented using myRIO® and the LabVIEW® software (National Instruments®). The angular displacement signals of the knobs are measured by the controller through the encoders. The controller then sets the displacement of the stepper motor in synchronous with the insertion control knob signal, and sets the steering coil's current in proportion to the steering knob signal. This system allows the user to remotely control the motion of the catheter simply from the two knobs without extensive training.

FIG. 7(b) shows the designed leg's vasculature phantom for the steering evaluation of the developed robotic catheter system. The phantom mimics the human leg's vasculature with two bifurcations (shown in FIG. 1). The bifurcations were 3D printed using the clear resin (Form 3, Formlabs) to provide a clear view of the catheter during remote steering. The bifurcations include two different Y-shape connections with 25° and 30° angles between two branches, with an outside diameter of 11 mm and inside diameter of 9 mm, and other dimensions shown in FIG. 7b. The 3D printed bifurcations were connected using clear tubing with an outside diameter of 8 mm and an inside diameter of 6 mm passing through the inner holes of the printed Y-shape connectors.

### Bending Performance of the Fabricated Catheter

To evaluate the bending performance of the fabricated catheter's distal segment and the magnetic actuation system and to validate the design procedures using FE-simulations described herein, current of varying amplitude was applied to the catheter's distal segment while measures its deflections. FIG. 8 shows the experimentally measured catheter tip displacement variation with respect to the current amplitude in the actuation coils (e.g. 4, 8 and 13 amperes), which is obtained by the images measured using the optical microscope and analyzed by a computer vision algorithm. The FE simulated catheter tip displacement data is also plotted in FIG. 8.

### Catheter Navigation Test in Leg's Vasculature Phantom

To evaluate the maneuverability and navigation capability of the proposed robotic system including the catheter, magnetic steering system, and the feed mechanism, the vasculature phantom and the insertion and bending degrees-of-freedom (DoF) control knobs in the control interface were utilized to remotely steer the catheter under the visual feedback provided by the digital microscope located above the Helmholtz coils. Of note, in a real surgical setting, this feedback could be replaced with interventional fluoroscopic images. In the performed experiments, a user was provided with the images from the digital microscope showing the catheters inside the vasculature phantom. Next, the user remotely controls the catheter to navigate through the vascular phantom including both the straight path and the shown bifurcations in FIG. 7b. FIG. 9 illustrates the photographs of apparatus steering, where the robotic catheter navigates into the right vascular branch of the bifurcation with a 25 degree angle. The process in FIG. 9 took 20 seconds.

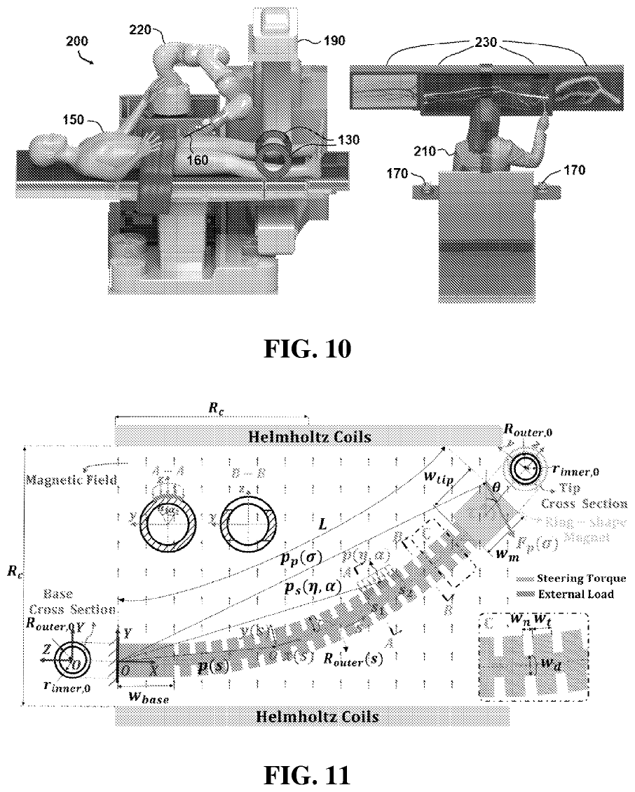

Referring now to FIG. 10, a robotic teleoperation system 200 is shown in which a surgeon 210 can remotely control and tele-operate catheter 160 using a robotic manipulator 220 while seeing the location of catheter 160 under X-ray (or other suitable visualization systems, including for example other types of radiographic imaging) via one or more visual display devices (e.g. monitors) 230. In the embodiment shown, robotic manipulator 220 controls insertion of catheter 160 while the steering is controlled with the magnetic actuation of one or more electromagnets 130 via controller 170 operated by surgeon 210. In particular embodiments, controller 170 may comprise one or more joysticks, a 3D mouse, or other suitable control mechanism. By remotely locating surgeon 210 a sufficient distance from patient 150, the risk of radiation for surgeon 210 can be removed.

### Mathematical Modeling

Additional analytical modeling and other aspects of exemplary embodiments of the present disclosure are provided below. As shown in FIG. 11, an exemplary embodiment of the present disclosure comprises a magnetically steerable robotic catheters (MSRC) with a flexural pattern composed of symmetric discrete notches, hollow instrument channel, a non-solid ring-shaped PM attached to the catheter's distal segment, and a magnetic actuation (i.e., Helmholtz coils). To establish a comprehensive analytical relationship between the planar deformation behavior of the catheter, magnetic field, steering torque, external loads and catheter's design parameters (i.e., flexure patterns, notch geometry, and catheter's size), an analytical modeling framework can be utilized. This mathematical framework can aid in understanding the importance of the mentioned variables/parameters during the design step as well as the performance of the catheter for the considered PAD application. Unlike recent literature (e.g., [35], [29]) that worked on model-based design of MSRCs, the proposed mathematical framework is derived based on Cosserat rod theory [36] and is more generic. This model can overcome the shortcomings of the existing structure (e.g., [35]), geometry (e.g., [29]), and design (e.g., [28]) specific models and can be applied for the design of MSRCs with a continuous or discontinuous cross-section geometry (i.e., with discrete flexural patterns).

In contrast to the existing algorithms proposed for solving the boundary value problem (BVP) formulation in Cosserat rod theoretical models (e.g., shooting method [38], [39]), a new formulation and iterative algorithm is proposed that does not require solving classic Cosserat rod differential equilibrium equations and their BVP formulations [37]. Of note, this can address the numerical stability issues when the second moment of area of the cross section functions (such as equation (4)) are not differentiable [37] in MSRCs with flexural patterns. An example catheter is shown in FIG. 1.

### Magnetic Actuation for the Steerable Robotic Catheter

The magnetic actuation of the proposed MSRC is shown in FIG. 1 in which Helmholtz coils—i.e., a pair of coils with distance equal to their radius [40]—are used to generate approximately uniform magnetic field between the coils [41]. Unlike most of the MSRCs in the literature utilizing a solid PM, a single ring-shape axially-magnetized PM is arranged at the catheter distal segment to not block the catheter's instrument channel. When currents are excited in the coils, steering torque is generated to the PM, which controls the direction of the catheter's distal segment. The magnetic flux density (MFD) generated by the Helmholtz coils can be obtained as [42]:

$$B = \frac{8}{5 \sqrt{5}} \frac{{μ}_{0} {N}_{c} ℐ}{{R}_{c}} \qquad ( 1 )$$

where μ₀=4π×10⁻⁷ T m/A is the permeability of free space, I is the current amplitude in the coils, N_{c} is the number of turns in each coil, and R_{c} is the coil radius.

Due to the small size of the utilized magnet, the ring-shapePM can be modeled as a magnetic dipole with a dipole moment m=BᵣVₘ/μ₀ [40], where Bᵣ is the remanence of the permanent magnet material and Vₘ is its volume. The steering torque generated on the magnet can then be calculated as:

$$τ = m \times B = mB sin θ \cdot e \qquad ( 2 )$$

where, as shown in FIG. 1, θ is the angle between the magneticdipole moment and the external magnetic field and e is the unit vector of the torque direction. With (1)-(2), the relationship between current I and steering torque τ can be found.

### Geometric Modeling of the Steerable Robotic Catheter

As shown in FIG. 11, the flexure pattern for the catheter distal segment is used to enhance the planar flexibility of the and enable it to readily steer through the vessels and particularly within the bifurcations. For the proposed catheter, we consider a flexure pattern consists of N notches evenly and symmetrically distributed on each side of the robot. Therefore, for such catheter we have:

$$L = {w}_{base} + {Nw}_{n} + ( N - 1 ) {w}_{t} + {w}_{tip} \qquad ( 3 )$$

where w_{base} is the length of proximal segment of the catheter and wₜᵢₚ is the length of its distal segment. Wₙ denotes the width of each notch, wₜ represents the distance between two adjacent notches, and wd denotes the width of the the MSRC's backbone (see FIG. 1). Of note, by adjusting these design parameters, the flexibility and steerability of the MSRC can be tuned and designed.

As shown in FIG. 11, unlike the MSRCs with a continuous cross sectional geometry [28], the proposed catheter in this study has a discontinuous cross section that varies along its arc length. Therefore, the second moment of area Iz(s) of the catheter's cross sections about the z axis can be defined using the following piecewise function:

$${I}_{z} ( s ) = \{ \begin{matrix}\begin{matrix}\frac{π E}{4} \cdot ( {R}_{outer , 0}^{4} - {r}_{inner , 0}^{4} ) \\ ⋃ [ {L}_{i} , {R}_{i} ] ⋃ [ L - {ω}_{tip} , L ]\end{matrix} & s ∈ [ 0 , {ω}_{base} ] \\ \begin{matrix}4 \cdot [ {\int }_{0}^{\frac{{ω}_{d}}{2}} {( {R}_{outer , 0}^{2} - {y}^{2} )}^{\frac{1}{2}} {y}^{2} dy \\ - {\int }_{0}^{\frac{{ω}_{d}}{2}} {( {r}_{inner , 0}^{2} - {y}^{2} )}^{\frac{1}{2}} {y}^{2} dy ]\end{matrix} & s ∈ ( {R}_{i} , {L}_{i + 1} ]\end{matrix} \qquad ( 4 )$$

where/is an integer and Lᵢ and Rᵢ are defined as: Lᵢ=w_{base}+ (I−1)wₙ+(I−1)wₜ for I∈[1, N] and Rᵢ=w_{base}+iwₙ+ (I−1)wₜ for I∈[1, N], respectively.

### Continuum Model of the Steerable Robotic Catheter

As shown in FIG. 11, the planar deformation behavior of the described MSRC in the preceding section can be described using the body frame pose (i.e., homogeneous transformation matrix T(s)∈SE(3)) along the midline arc length parameter s∈[0,L] with respect to the global frame {XYZ} as follows

$$T ( s ) = ( \begin{matrix}ℛ ( s ) & p ( s ) \\ {0}^{T} & 1\end{matrix} ) \qquad ( 5 )$$

where L is the overall length of the catheter's midline, p(s)∈R³ denotes the position vector, and R(s)=[x(s),y(s),z(s)]∈SO(3) represents the orientation matrix of the body frame {xyz}. X(s)∈R³ is the unit vector tangent to the catheter's midline. Unit vectors y(s)∈R³ and z(s) ∈R³ of the body frames lie on the plane of the catheter's cross-sections.

The continuous evolution of the body frame configuration along the arc length parameter s can be represented using the angular and linear rate of changes of configuration T(s)∈SE(3) with respect to s (i.e., the twist vector V_{b}(s)=[ω_{b}(s),v_{b}(s)]T∈R⁶) [37]:

$$\dot{T} ( s ) = T ( s ) [ {V}_{b} ( s ) ] \qquad ( 6 )$$

$$\dot{T} ( s ) = ( \begin{matrix}ℛ ( s ) & p ( s ) \\ {0}^{T} & 1\end{matrix} ) ( \begin{matrix}[ {ω}_{b} ( s ) ] & {v}_{b} ( s ) \\ {0}^{T} & 0\end{matrix} ) \qquad ( 7 )$$

where dot represents the derivative with respect to s throughout this paper. [Vb(s)]∈se(3) is the skew-symmetric matrix representation of the twist vector Vb(s)∈R³. Vb(s)∈R³ denotes the linear rate of change and [ωb(s)]∈so(3) is the skew-symmetric matrix representation of the angular rate of change ωb(s)∈R³.

### Modeling External Loads Acting on the Catheter

During steering of a MSRC through the leg vasculature, a MSRC may experience external point and distributed surface loads due to the interaction with the environment. Therefore, it is very critical to consider the effect of these loads on the deformation behavior and steerability of the catheter.
3) Modeling Interaction Point Loads: As shown in FIG. 11, we may consider a general external point load Fp(σ) with an arbitrary magnitude and direction applied along any point on the surface of the MSRC with a known location pₚ(σ), where σ represents the location along the arc length parameter s.
   The moment mₚ(s) generated by this point load along the arc length parameter s can be calculated as follows [43]:

$${m}_{p} ( s ) = \{ \begin{matrix}{r}_{p} ( s , σ ) \times {F}_{p} ( σ ) & s ∈ [ 0 , σ ] \\ 0 & s ∈ ( σ , L ]\end{matrix} \qquad ( 8 )$$

where rₚ(s,σ)=p(s)−pₚ(σ) is the arm vector.

### Modeling Interaction Distributed Surface Loads

As shown in FIG. 11, the surface of the MSRC can be parameterized using η representing the location along the arc length parameter s and angle α defining the amount of revolution about x(s) axis. Using these parameters, a general distributed surface load/pressure can be defined using f(η, α) with an arbitrary magnitude and direction acting at the location pₛ(η, α) on the MSRC's surface, where η∈[s1, s2] and α∈[α1, α2]. The differential external force dFₛ(η, α) due to distributed surface load/pressure f(η, α) can be expressed as dFₛ(η, α)=f(η, α) dA(η, α) where dA(η, α)=Rₒᵤₜₑᵣ(n)dηdα is a small element of the MSRC's surface area. The moment mₛ(s) generated by this distributed surface load/pressure f(η, α) along the arc length parameter s can also be obtained using:

$${m}_{s} ( s ) = \{ \begin{matrix}{\int }_{{D}_{1}} {r}_{s} ( s , η , α ) \times f ( η , α ) dA ( η , α ) & s ∈ [ 0 , {s}_{1} ] \\ {\int }_{{D}_{2}} {r}_{s} ( s , η , α ) \times f ( η , α ) dA ( η , α ) & s ∈ ( {s}_{1} , {s}_{2} ] \\ 0 & s ∈ ( {s}_{2} , L ]\end{matrix} \qquad ( 9 )$$

where rs(s, η, α)=p(s)−ps(η, α) is the moment arm vector. Domains D1 and D2 are defined as: D1={A(η, α): s₁≤η≤s₂; α₁≤α≤α₂} and D2={A(η, α): s₁≤η≤s; α₁≤α≤α₂}.

### Equilibrium Equations of the Coupled Physical System

Classical forms of Cosserat rod differential equilibrium equations [36] are widely used in modeling continuum manipulators (e.g., [37] and MSRCs (e.g. [30]). Despite the generality of these differential equilibrium equations, they may experience numerical stability issues when differentiating the second moment of area of the cross section functions [37] (such as equation (4) for modeling catheters with flexural patterns and/or discontinuous cross section geometry (e.g., the robot shown in FIG. 11 and described in the Section titled “Geometric Modeling of the Steerable Robotic Catheter”. Therefore, to address such numerical stability issues, we have developed the following formulations and solution algorithm described in the following section entitled “Iterative Algorithm and Convergence Analysis.”

Considering a generic MSRC shown in FIG. 11, the relationship among internal loads exerted by the catheter's elasticity, external steering torque τ(s), external point Fp(s) and distributed surface loads f(η, α) can be expressed using (2), (8), (9) and the following static equilibrium equation:

$${K}_{w} ( s ) {ω}_{b} ( s ) = τ ( s ) + {m}_{p} ( s ) + {m}_{s} ( s ) \qquad ( 10 )$$

where the stiffness matrix K_{w}(s) can be defined as K_{w}(s)=EI_{z}(s) for the case of the considered catheter, described in the section entitled “Geometric Modeling of the Steerable Robotic Catheter”, with the Young's modulus E. However, as mentioned, the modeling framework is generic and can be applied to catheters with other flexural pattern geometries simply by updating the stiffness matrix K_{w}(s). Of note, in this equation, since the ring-shape magnet is tightly attached to the distal segment of the catheter shown in FIG. 11, it can be considered as a rigid segment (i.e., ω_{b}(s)=0 for s∈(L−wₘ,L] in which wₘ is the length of the ring-shape magnet). Therefore, the applied forces/moments only can influence on the segments before the magnet's location (i.e., s∈[0,L−wₘ]).

### Iterative Algorithm and Convergence Analysis

As shown in (2), (5), and (10), magnetic steering torque π of the catheter depends on the angle θ between the magnetic dipole moment and the external magnetic field or catheter's deformation behavior (e.g., tip orientation) while catheter's deformation behavior itself also depends on the steering torque. Therefore, in the proposed multiphysical model, steering torque and catheter's deformation behavior are correlated and both unknown. Additionally, due to the discontinuous cross sections of the catheter along its length, the typical solving techniques of Cosserat rod models relying on classic differential equilibrium equation and their BVP formulations [37] might not be an optimal choice. These factors may make solving the proposed mathematics model very challenging. To address these challenges and considering known catheter design parameters and the input current passing through the Helmholtz coils, an iterative solution approach is proposed as summarized in Algorithm 1 below. For the convenience of analysis, it is assumed only steering torque is applied to the catheter and there is no interaction between the catheter and environment (i.e., Fₚ(σ) and f (η, α) are set to zero). It is also assumed the catheter is initially unbent and has a straight configuration and therefore can initialize the algorithm with angle θ=π/2. Considering sin θ⁰=sin π/2=1 and equations (2), (5), (7), and (10), this initialization would result in the maximum possible steering torque in the next iteration (i.e., τ¹) and therefore maximum possible deformation behavior for the catheter ω¹. Of note, utilizing these maximum values in (10) and (8) would lead to the minimum possible angle between the magnetic dipole moment and the external magnetic field in the first iteration. Subsequently, in the next iteration, utilizing the minimum possible angle θ¹ will result in the minimum possible τ² and ω² and maximum possible angle θ². T², ω² are then used to calculate the second largest possible steering torque τ³, the second largest possible deformation behavior of the catheter ω³ and the second smallest possible angle θ³. Following the procedure, the third smallest possible steering torque τ⁴, the third smallest possible deformation behavior of the catheter ω⁴ and the third largest possible and the third largest possible angle θ⁴ and so on can be found iteratively. Subsequently, when k goes to infinity, θ, τ, and ω_{b} will converge to a fixed point value θ*, τ* and ω* [44], which are indeed the solution of the equilibrium equation (10). It is worth noting that the iteration process is terminated when the increment between the calculated MSRC's positions in two successive iterations are less than a user-defined threshold value ∈.

Algorithm 1: Solving the Multiphysical Model

| | Input: [symbol] , m, E, R_{outer,0}, r_{inner,0}, w_{base}, wₜᵢₚ, wₙ, | |
|---|---|---|
| | wₜ, N, θ⁰ rₚ⁰, rₛ⁰, Fₚ, f; | |
| | Initialization: | |
| 1 | B ← Calculate MFD (1) [ [symbol] ]; | |
| 2 | I_{z} ← Calculate the second moment of area (4) | |
| | [R_{outer,0}, r_{inner,0}, w_{base}, wₜᵢₚ, wₙ, wₜ, N]; | |
| 3 | k ← 0; | |
| | Iterative Process: | |
| 4 | do | |
| 5 | \| | τ^{k+1} ← Calculate steering torque (2) [B, m, θ^{k}]; |
| 6 | \| | mₚ^{k+1} ← Calculate moment due to external point |
| | \| | load (8) [rₚ^{k}, Fₚ]; |
| 7 | \| | mₛ^{k+1} ← Calculate moment due to distributed |
| | \| | surface load (9) [rₛ^{k}, f]; |
| 8 | \| | ω_{b}^{k+1} ← Solve equilibrium equation (10) [E, I_{z}, |
| | \| | τ^{k+1}, mₚ^{k+1}, mₛ^{k+1}]; |
| 9 | \| | T^{k+1} ← Solve ODE equation (7) [ω_{b}^{k+1}]; |
| 10 | \| | [p^{k+1}, [symbol] ^{k+1}, rₚ^{k+1}, rₛ^{k+1}] ← Find position |
| | \| | vector and orientation matrix (5) [T^{k+1}]; |
| 11 | \| | θ^{k+1} ← Calculate the angle between the |
| | \| | magnetic dipole moment and the external |
| | \| | magnetic field [ [symbol] ^{k+1}]; |
| 12 | \| | k ← k + 1; |

| | |
|---|---|
| 13 | while ∥p^{k+1}(s) − p^{k}(s)∥ < ϵ; |
| | Output: p, [symbol] , θ, τ |

### Simulation and Experimental Validations

### Simulation Studies

To evaluate the performance of the proposed analytical model and the solution algorithm, we conducted three types of simulations in MATLAB (MathWorks, Inc.) on MSRCs with geometries described in Section II-C and shown in FIG. 11. The considered catheters of the three simulations share the following identical design parameters: L=50 mm, R_{outer,0}=1 mm, and r_{inner,0}=0.75 mm, as defined in FIG. 1. The Helmholtz coils and magnetic field are set with the parameters: μ₀=4π 10⁻⁷ T m/A, N_{c}=200 mm, R_{c}=200 mm, Bᵣ=2.5 T. The ring-shaped magnet has a 3 mm outer diameter and 1.5 mm inner diameter with an axial length of 4 mm. Of note, the selected MSRCs parameters are within the ranges of catheters used for PAD applications. Nevertheless, as mentioned, the proposed modeling framework is generic and the used parameters can be changed depending on the application.

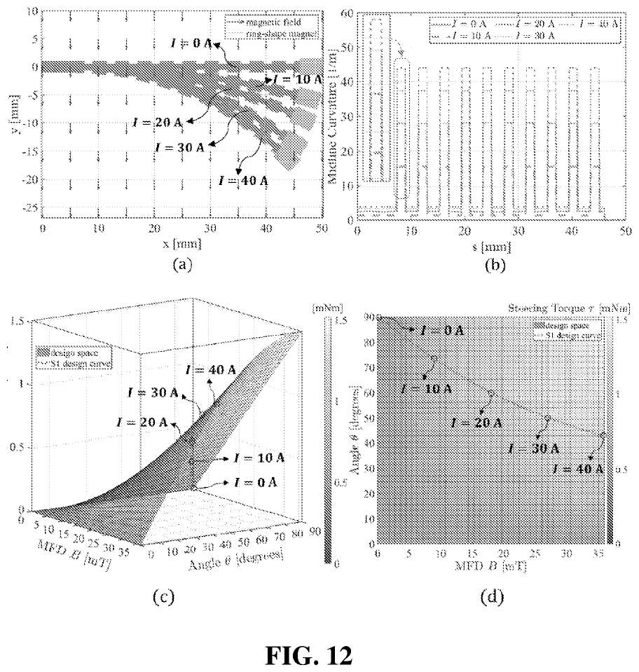

(1) Input Current Analysis: In the first type of simulations, we investigated the influence of changing the input current passing through Helmholtz coils on various parameters including: deformation behavior of MSRCs, the midline curvature distribution, the MFD B, the angle between the magneticdipole moment and the external magnetic field θ, and the steering torque t. This simulation was performed using the catheter of scenario S1 with input current ranging from I=0˜40 A with a 10 A increment. Geometrical parameters of the considered flexure pattern for this catheter are detailed in Table I. FIG. 12(a) compares the deformation behaviors of the selected MSRC S1 under five different input currents. FIG. 1 2(b) illustrates the obtained midline curvature distributions along arc length parameter s for the considered five different currents. More importantly, FIG. 12(c) represents a “design space”, generated by equation (2), demonstrating the space of all possible steering torques τ, MFDs B, and angles θ when the input current is changing from =0 40 A and the motion constrain cause by the MSRC on the PM's motion is neglected and the PM can freely float between the Helmholtz coils (i.e., PM is not attached to the catheter). Itis worth emphasizing that for a PM attached to the distal end of a catheter, motion of the PM magnet within the Helmholtz coils is constrained by the deformation behavior of the catheter. In this case, the aforementioned “design space” of τ, B, and θ will be reduced to a “design curve” due to the additional motion constrain imposed from the MSRC's deformation behavior on the PM's motion. Of note, this curve is generated by solving equation (10), which considers both the magnetic actuation forces and the deformation behavior of the MSRC. This curve also shows the corresponding points that matches with the simulated current scenarios in FIG. 12(a). It is worth emphasizing that the “design curve” is always a subset of the “design space” and lies on the surface of the “design space”. FIG. 12(d) also gives a 2D view of FIG. 12(c).

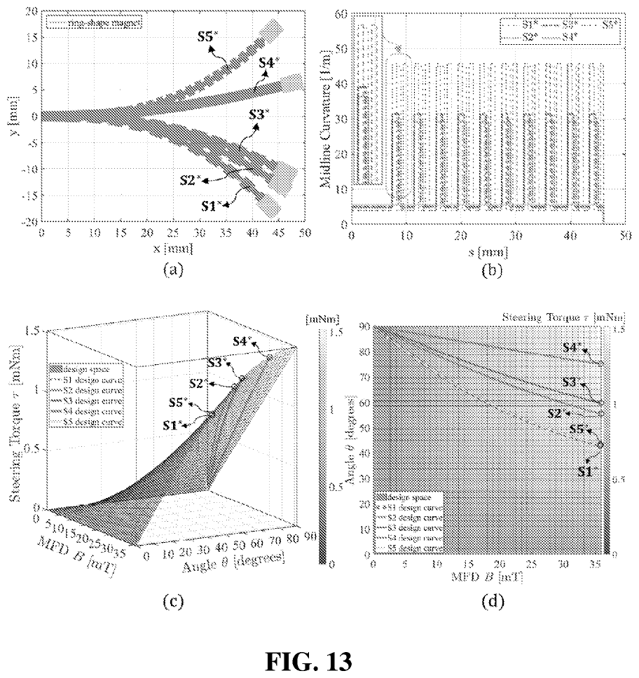

(2) Flexural Pattern Analysis: The second type of simulations were performed to investigate the effects of discontinuous cross-section geometries or flexure patterns of the five selected scenarios (i.e, S1˜S5) on the deformation behavior of an MSRC, the midline curvature distribution, and the relation-ships among MFD B, the angle θ, and the steering torque τ. Geometrical parameters of the considered flexure pattern for each catheter of the five scenarios have been detailed in Table I. FIG. 13(a) compares the deformation behaviors of the five different scenarios S1*˜S5* under the arbitrarily selected input current (i.e., I=40 A). FIG. 13(b) illustrates the curvature distribution along catheter midline. FIG. 13(c) shows the “design space” together with a comparison of the “design curves” associated with S1˜S5. FIG. 13(d) also provides a 2D view of FIG. 13(c). Of note, “*” represents a scenario under the specific input current I=40 N.

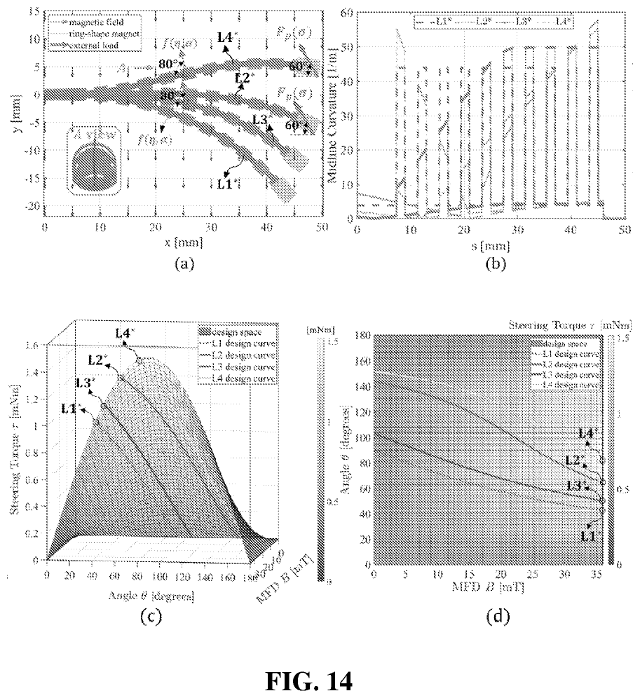

(3) External Load Analysis: The third type of simulations investigates the impact of external loads (i.e., four considered external loading scenarios L1*˜L4*) on the deformation behavior of an MSRC, the midline curvature distribution, the magnetic flux density B, the angle between the magnetic dipole moment, the external magnetic field θ, and the steering torque t using flexure pattern of scenario S1. Parameters of the four considered point and distributed external loading scenarios L1*˜L4* are shown in Table II. FIG. 13(a) compares the deformation behaviors of the four considered external loading scenarios L1* L4* under the selected input current (i.e., I=40 A). FIG. 14(b) compares the curvature distribution along MSRC midline for these studies. FIG. 14(c) shows the design space and corresponding “design curves” of the four considered external loading scenarios L1˜L4. FIG. 14(d) also gives a 2D view of FIG. 14(c). Of note, “*” represents a scenario under the specific input current I=40 N

### Experimental Studies

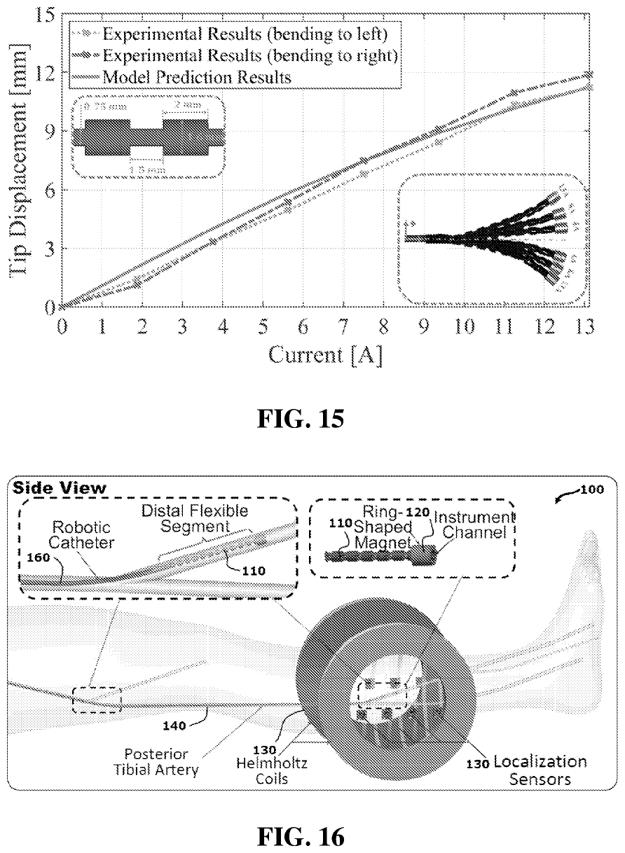

To experimentally evaluate the performance of the proposed modeling framework and solution algorithm, we designed and fabricated a MSRC with its complementary magnetic actuation system. FIG. 7 shows the overview of the experimental setup comprising of a magnetic steering system, the designed MSRC with a flexible distal segment and the ring-shape magnet adhered at its distal end, a catheter feeding mechanism, a controlbox as the remote operation interface, a digital microscope simulating the fluoroscopic images in a real PEI and provides the visual feedback on the MSRC. The magnetic steering system consists of a pair of custom-fabricated Helmholtz coils to create a uniform magnetic field for steering of the MSRC. The Helmholtz coils have a diameter of 220 mm and are configured with a distance of 110 mm. The coils are connected in series and are driven by a full-bridge bi-directional switching-type power amplifiers (B07NP6XNPR, Cytron Technologies©) with 20 A current capacity. The coils have 120 turns each and under the maximum allowed current amplitude of 13 A (limited by the power supply being selected) can generate a steering field of 14.8 mT. Based on the the proposed model and the PAD application requirements, we designed and fabricated a MSRC (using heat shrink tubing (40142000, MILAPEAK) with Young's modulus E=0.219 Gpa) with a flexure pattern that can create 12 mm maximum deflection at the MSRC's distal end with the following flexure pattern details defined in FIG. 11: wₙ=1.5 mm, wₛ=2 mm, w_{d}=0.75 mm, w_{base}=1 mm, wₜᵢₚ=6 mm, N=8, R_{outer,0}=0.8 mm, and r_{inner,0}=0.5 mm. Of note, this design provides adequate deflection for remote steering of the catheter and passing through the leg's vasculature bifurcations. The fabricated flexible segment then adhered to a commercially available catheter (G11430, Cook Medical, USA) with outer diameter of 1.5 mm from its proximal end. A ring-shaped magnet (N52 grade NdFeB, K&J Magnetics Inc.) with ⅛ inch outer diameter, 1/16 inch inner diameter, and ⅛ inch axial length was also adhered to the distal end of the flexible segment (see FIG. 7 panel (a)) As shown in FIG. 7 panel (a) the catheter feeding mechanism uses a NEMA 17 stepper motor and implements the friction driven design where the catheter's insertion motion is driven by the friction force between the drive wheel and the catheter. The remote control interface consists of a box housing two control knobs driving two optical encoders (Taiss Inc., 600 counts/revolution), a myRIO em-bedded controller (National Instruments), and a stepper motor driver (Stepperonline). One knob is controlling the catheter insertion motion, and the other is controlling the steering of the catheter distal segment. The system is controlled using a real-time controller implemented using myRIO and the LabVIEW software (National Instruments). To evaluate the bending performance of the fabricated catheter's distal segment and the magnetic actuation system and compare it with the proposed multiphysical model, we applied current of varying amplitude (0˜13 A with 2 A increments) to the Helmholtz coils and measured the planar deformation of the distal segment on two different directions. FIG. 15 compares the tip displacement variation with respect to the current amplitude in the actuation coils between experimental results and the proposed model prediction.

### Discussion

As observed in FIGS. 12 (a) and (b), when the input current increases, the MSRC experiences a higher deformation behavior and midline curvature distribution, respectively. As shown, the notched section of the catheter bends more than the tubular section because of the smaller second moment of area of these sections (see (4). Also, as mentioned, due to the flexural pattern of the MSRC, a discrete curvature is experienced along the length of robot, which can cause numerical instability issues when using the classical modeling approaches (e.g., [37]). The “design space” shown in FIG. 12(c) and FIG. 12(d) clearly demonstrates the correlation between all the involved parameters in the proposed multiphysical model. As can be seen from the “design curve”, for the considered flexural pattern, with the increase of current, θ is monotonically decreasing with its minimum happening at I=40 A and θ=45 degrees.

In FIG. 13(a) and FIG. 13(b), despite that identical input current is used for the considered five flexural geometries S1*˜S5*, distinct deformation behaviors and midline curvature distribution are observed, which clearly demonstrates the influence of these geometries on the performance of an MSRC. For instance, as shown in FIG. 13(a), if we increase the width of the MSRC backbone ω_{d} from 1 mm for S1* to 1.2 mm for S2*, the catheter maximum deformation will decrease. For an extreme case (i.e., S4*) in which we do not consider a notch pattern (i.e., zero notch width) and increase the width of the backbone to 4 mm, the minimum deformation behavior is experienced. Although, notch number in S5* design is twice of S1*, they create almost identical tip displacement (i.e., 18 mm), highlighting the importance of utilizing the proposed modeling approach as a design tool and before the fabrication procedure. Of note, fabrication of S5* design requires more precision and is more time-consuming than S1* design while offering a similar maximum deformation behavior. As shown in FIG. 13(c) and FIG. 13(d), although five selected geometries S1˜S5 are distinct, their corresponding “design curves” all lie on the “design space” and similar to the previous simulation studies are a subset of the design space. Similar to the results shown in FIG. 13(a), maximum deformation behavior (i.e., angle θ=45 degree) are also for S1* and S5*. In other words, based on the demonstrated “design space” and “design curve”, with the considered flexural patterns and MSRCs parameters, we never can achieve more than 18 mm of tip displacement and less θ=45 degree angle.

As shown in FIG. 4(a) and FIG. 4(b), for MSRCs with identical flexural patterns (i.e., S1) and input current (i.e., I=40 A), different external loading conditions create distinct deformation behaviors and midline curvature distribution. As shown, applying external loads to the catheter body in an op-posing direction of the steering torque reduces the deformation of an MSRC. In FIG. 14(a), compared to the L1* case with no external loads, external point load Fₚ(σ) acting on the edge of the ring-shaped PM (i.e., L2* case) has a greater influence on reducing the deformation behavior of the MSRC (i.e., 14 mm less tip deflection) compared to a distributed load acting on the middle of MSRC (i.e., L3* case). Due to the effect of external loads, the curvature distribution of these simulation scenarios (shown in 4(b)) are also very different compared to FIG. 12(b), and FIG. 12(b). The maximum curvature value occurs at a point near the catheter's tip segment where external point load Fₚ(σ) and distributed surface load f(η, α) are both applied to the catheter body in FIG. 14(b). Unlike the effects of the catheter's geometry design and flexure pattern in FIG. 13, the effects of external loads clearly change the locations of the starting point of the “design curves” in FIG. 14(c) and FIG. 14(d) where the angle θ is over 90 degrees and the MFD B is small. As can also be seen in FIG. 4a, this is mainly caused by the external loads creating opposite bending directions for the MSRC as compare to the free bending cases. However, as B increases (or input current increases), the steering torque overcomes this opposing external loads and the MSRC bends back to the downward direction, which explains why the angle θ decreases in this process. Similar to the other design and curve spaces, FIG. 14(c) and FIG. 14(d) can clearly demonstrate the performance of the MSRC in different loading and steering conditions.

As shown in FIG. 15, the maximum experimentally obtained MSRC tip deflection (i.e., 11.24 mm) at 13 A matches perfectly with the prediction results of the proposed model prediction. Additionally, the calculated root mean square error (RMSE) and the mean absolute error (MAE) between the simulations and the experimentally obtained tip displacements under the applied input currents are 0.52 mm and 0.48 mm, respectively. This clearly validates the accuracy of the proposed model and the solution algorithm for modeling a complex multiphysical system.

In this disclosure, a novel and generic multiphysical framework and iterative solution algorithm were proposed that can model MSRCs with flexure patterns to be used for PAD treatment. We also proposed the concepts of “design space” and “design curve” to completely study the unknown correlation between the input current in Helmholtz coils, magnetic field, steering torque, external loads, flexure patterns, external interaction loads and deformation behavior of the catheter without relying on the classic Cosserat rod differential equilibrium equations and their BVP formulations. These spaces provide an intuitive understanding of the correlation between the aforementioned parameters and can serve as an effective design and optimization tool for tuning steerability of MSRCs and the magnetic actuation system. The conducted experimental results (RSME=0.52 mm and MAE=0.48 mm) demonstrated the accuracy of the proposed model and algorithm in predicting the tip displacement of an MSRC. Although in this study, we mainly focused on flexure patterns of catheters that enable planar deformation in this study, the proposed approach and robotic system can be readily extended for modeling and evaluations of MSRC's bending in 3D as our future direction.

In summary, exemplary embodiments of the present disclosure include the design and fabrication of a magnetically steerable robotic catheter with flexible distal segment and an open instrument channel to mitigate the existing challenges while steering catheters for treatment of PAD. For instance, as opposed to the typically-used solid magnets in the literature (e.g., [43]), exemplary embodiments include a ring-shaped magnet to provide the clinicians with an open space to deploy required implants or instruments. Additionally, to improve steerability of the current catheters, exemplary embodiments include a fabrication procedure to design a flexible distal segment that can be adhered to a conventional catheter. The disclosed fabrication method can be replaced with the conventional complex and costly fabrication procedures such as EDM and laser cutting to manufacture precise flexure mechanisms on medical devices (e.g., [44]). The disclosed approach took advantage of the thermoplastic property of heat shrink tube materials to enhance the cutting precision of miniature flexure patterns. Accordingly, it is possible to utilize a manual swivel knife to fabricate miniature square patterns with feature size below 1 mm, shown in FIG. 6 and FIG. 8. The comparison results—shown in FIG. 8—verifies the accuracy of the proposed cost-effective procedure. It is worth noting that although the disclosed embodiment utilizes identical square patterns, other embodiments may use different configurations to fabricate other types of flexural patterns such as triangular and/or variable-dimension patterns.

The performance of the proposed system was evaluated via magnetically actuated bending tests where the Helmholtz coils are excited to generate external steering field up to 25 mT. For the designed flexure pattern, the maximum achieved catheter tip bending displacement, similar to the FE simulations, was 12 mm. Of note, this maximum bending was designed based on the required maximum displacement for passing through the leg bifurcations, shown in FIG. 1 toward treatment of PAD. As shown in FIG. 8, the calculated tip bending displacement of the catheter in FE simulations was compared to the experimentally obtained results. The calculated root mean square error between the FE simulations and the experimentally obtained tip displacements were calculated as 0.575 mm and 0.328 mm for bending to the left and right side of the catheter, respectively. This clearly validates the numerical FE models and the performance of the catheter and the magnetic system.

With the disclosed fabrication procedure and verified FE simulations, it is possible to optimize and fabricate miniature flexure patterns and the parameters of the magnetic steering actuation mechanism to satisfy the design requirements and medical needs. The navigation test demonstrated the effectiveness of the disclosed robotic system to steer and insert the robotic catheter in a leg vasculature phantom under remote operation. This is of paramount importance to minimize the amount of radiation exposure for both patients and clinicians and improve safety of the procedure.

As shown above, the magnetic and mechanical models for the robotic catheter are validated by experimental measurements. Certain embodiments can provide optimization for the catheter distal segment via fully integrated multi-physical simulations to maximize the steerability of the catheter's distal segment. Particular embodiments can also include a robotic catheter design that enables out of plane bending to have a full three-dimensional workspace. Certain embodiments can also comprise custom-designed ring-shape magnets that will enable the use of proposed robotic catheter system in vasculatures with very small size.

With an embedded magnetic source at the tip, the disclosed magnetically steerable robotic catheter has the potential to enable the use of magnetic field for tip localization, which provides essential position feedback signals for the catheter to navigate through the complex vascular systems. Specific embodiments can also be integrated with a magnetic localization system. Such high speed yet low spatial-resolution magnetic positioning signal will complement the high 32pecial-resolution, low frequency signal from interventional fluoroscopic images under X-ray radiation, and has the potential to further reduce the X-ray exposure to both patients and the radiologists [50].

Despite various advantages of today's MSRC systems, the location sensing for the catheter within a vessel still highly depends on fluoroscopic imaging. However, this imaging modality can put clinicians and patients at risk of increased x-ray radiation exposure, particularly during lengthy procedures, highlighting the need for developing new radiation-free sensing technologies. In MSRCs, a small permanent magnet (PM) is typically arranged at the tip of the catheter to allow for magnetic steering and/or insertion [52], [57]-[61]. This design offers the exciting possibility of using the magnetic field from the catheter tip for location sensing. Developing an accurate and reliable magnetic localization solution for MSRCs can significantly reduce the fluoroscopic x-ray utilization for catheter navigation, thereby reducing the x-ray exposure and procedural length for these operations.

Magnetic localization, where the magnetic field from a PM or a coil is measured and used to estimate its location and orientation, has demonstrated excellent performance in various applications including industrial robot calibration [62], robotic capsule endoscopes [63]-[65], miniature robots [66], [67], and continuum manipulators [68]. Despite these successes, several major technical barriers must be tackled to enable the magnetic localization technology to be successfully used for MSRC systems used in PAD interventions: (i) The catheter must fit within the vessel lumen and thus must have a smaller diameter profile (of 3 mm or smaller), which is significantly smaller than the PM size being used in the prior art. This fact can lead to noises in the sensor signals and thus estimation inaccuracy. Highly sensitive sensors and de-noise algorithms must be used to enable the effective estimation of the catheter location. (ii) The accuracy of the magnet localization directly depends on the accuracy of sensor locations and the model for the PM, which are typically assumed to be known a priori. To address these uncertainties, data-driven calibration algorithms for sensor locations and PM dipole moment have been developed [69]. However, in prior studies, the calibrations of various parametric uncertainties are typically conducted sequentially, which can result in sub-optimal calibration results. New calibration algorithms that address the coupling between the estimation for various uncertainties must be developed to attain improved magnetic localization accuracy. (iii) In MSRC systems, a strong external magnetic field is needed to actuate the catheter's steering, which is typically from actuation coils [69], [70] or a large piece of PM [52]. Such an actuation field can saturate the magnetic sensors being used for localization.

New algorithms are needed to enable the integration of magnetic actuation and magnetic location sensing to minimize the required duration of fluoroscopic imaging under this limitation.

In summary, magnet position estimation requires knowing (a) magnet strength M (scalar) and (b) the sensors' exact location pₛ (vector of (3N) x1, and N is the number of sensors), and errors in these parameters can lead to magnet location error. Unfortunately, errors exist in magnet strength and sensor locations. These errors can be identified through a calibration process, which put the magnet at a series of known positions and collects the data, and then solves for the magnet strength and sensor location parameters by a least square problem. This least square problem solves for the M and ps that minimizes the discrepancy between magnet location estimated using the M and p values and the known location. Typical existing solutions complete the calibration for M and ps either individually or sequentially, which leads to sub-optimal results due to the interdependency of the two parameters. To resolve this interdependency issue, M and ps should be calibrated simultaneously, but this is not feasible computationally. To provide a computationally effective approach to solve this interdependency issue, a nested approach is disclosed herein which provides optimality and can achieve better calibration result than typical existing systems.

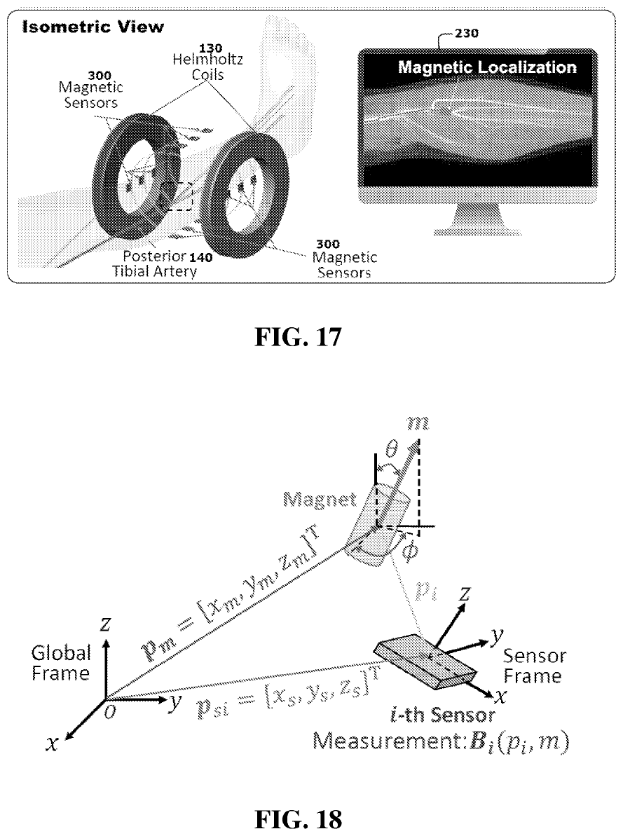

Referring initially to FIGS. 16 and 17, apparatus 100 is similar to previously described embodiments, but further comprises a plurality of magnetic sensors 300 configured to detect a location of a magnetic element. FIG. 16 illustrates a side view of apparatus 100, while FIG. 17 illustrates an isometric view of apparatus 100. In this embodiment, magnetic sensors 300 are coupled to an electromagnet 130 (e.g. Helmholtz coils) and are configured to detect a location of permanent magnet 120 coupled to segmented tubular 110 as part of a magnetically-steerable catheter 160, as previously described herein. It is understood, however, that in other embodiments, magnetic sensors 300 may be used to detect a position of a magnetic element in other applications, including for example, other medical instruments or any system which can utilize non-invasive imaging technologies.

As explained in further detail below, during use of apparatus 100 magnetic sensors 300 can be used to detect a position of permanent magnet 120 as catheter 160 is inserted into an artery 140. By utilizing magnetic sensors 300 to detect the position of permanent magnet 120 (rather than X-ray imaging) the patient and medical personnel are exposed to less radiation.

Accordingly, exemplary embodiments of the present disclosure are able to address the above-mentioned limitations and enable the use of magnetic localization for MSRC systems as a means to reduce x-ray radiation in medical procedures including, but not limited to, PAD interventions. Specific embodiments comprise a catheter with a ring-shaped permanent magnet (3 mm OD, 1.6 mm ID, 6 mm length) arranged at its tip for magnetic steering. In particular embodiments, magnetic sensors 300 are configured as a cylindrical array of highly-sensitive magnetoresistive sensors. Certain embodiments also utilize a low-latency magnet location estimator based on the Levenberg-Marquadt (LM) algorithm to track the location of the catheter tip in real-time.

Particularly, to enable improved estimation accuracy while maintaining computational simplicity, a novel nested calibration algorithm for sensor positions and magnetic strength is provided in exemplary embodiments. Accordingly, exemplary embodiments of the present disclosure provide a novel integration method for magnetic actuation and magnetic location sensing in systems (including, for example MSRC systems), where fluoroscopic imaging is only required during catheter steering at bifurcations in the vasculatures. Experiments show that an average position estimation error of 1.27 mm can be achieved after calibrating the system using the proposed algorithm. Exemplary embodiments can successfully navigate into the desired branch of bifurcation with visual feedback using magnetic localization during insertions and a digital microscope during steering, which shows the excellent potential of the proposed framework for reducing x-ray imaging in practical interventions. Compared with recent literature on magnetic localization [68]-[73], exemplary embodiments of the present disclosure provide improved performance in terms of magnet volume/sensor-to-magnet distance ratio [mm³/mm] and localization accuracy, as shown in FIG. 17. Embodiments of the present disclosure provide significant benefits to medical personnel and patients by integrating magnetic actuation and magnetic localization for surgical robot applications.

### Mathematical Model

FIG. 18 illustrates a diagram for the variables used in a magnet localization algorithm according to one embodiment. Since PM in an MSRC is small and magnetic sensors are placed sufficiently far away from the magnet, the PM's flux density at i-th sensor location Bi∈R³ can be described by the dipole model [74] as:

$${B}_{i} = \frac{{p}_{0}}{4 π} ( \frac{3 ( {m}^{T} {p}_{i} \cdot {p}_{i} )}{{‖ {p}_{i} ‖}^{5}} ) - \frac{m}{{‖ {p}_{i} ‖}^{3}} ) , \qquad ( 1 )$$

where m∈R³ is the magnetic dipole moment vector of the PM, μ₀ is the magnetic permeability of free space, and pᵢ ∈R³ is the vector from the center of the magnet to i-th sensor location. The vector pᵢ ∈R³ can be calculated via

$${p}_{i} = {p}_{s} i - {p}_{m} \qquad ( 2 )$$

where pₛ_{ᵢ}=[xₛ,yₛ,zₛ]^{T} and pₘ=[xₘ,yₘ,zₘ]^{T} are the coordinates of i-th sensor and magnet in the global-fixed frame, respectively. The dipole moment vector m∈R³ for the PM can be represented as

$$m = M ( sin θ cos ϕ i + sin θ sin ϕ j + cos ϕ k ) , \qquad ( 3 )$$

where i, j, and k are the unit vectors in the x, y, and z-axes, respectively; θ and ϕ are the angles of magnet's orientation in a spherical coordinate with its origin at the center of the magnet location, as shown in FIG. 18; M represents the dipole moment strength of the PM, which can be calculated as:

$$M = \frac{{B}_{r} {V}_{m}}{{μ}_{0}} , \qquad ( 4 )$$

where Bᵣ is the magnetic remanence of the PM material, and
Vₘ is the volume of the magnet.

Assuming the magnetic sensor can measure the magnetic flux density at the sensor location along three perpendicular axes in its sensor-fixed frame, the measured flux density in the sensor frame ^{s}Bᵢ can be calculated as

$${s}^{} {B}_{i} = {T}_{i} {B}_{i} , \qquad ( 5 )$$

where Tᵢ∈R^{3×3} is the rotation matrix between the global coordinate and the i-th sensor-fixed coordinate. All vectors with superscripts “s” represent coordinates in a sensor-fixed frame, while those without a superscript are defined in the global frame.

### Magnetic Localization Algorithm

This section introduces the magnetic localization algorithm used for the MSRC tip location sensing, which is based on the algorithm introduced in Taylor et al. [71] and is modified to suit the MSRC application.

Based on the dipole model introduced in the section entitled “Mathematical Model”, a nonlinear optimization problem can be formulated and solved to estimate the magnet's position and orientation as

$$ξ = {arg min}_{ξ} {\sum }_{i = 1}^{{N}_{a}} {‖ {s}^{} {B}_{i} ( ξ , {p}_{si} , M ) - {s}^{} {B}_{i}^{′} ‖}^{2} , \qquad ( 6 )$$

where ξ\hat{ }=[p\hat{ }ₘ^{T},θ\hat{ },ϕ\hat{ }]^{T} ∈R⁵ is a vector of the estimated position and orientation angles for the magnet, Ns is the number of sensor measurements available for the magnetic location estimation, and ^{s}B′ᵢ is the measurement data from i-th sensor in its individual sensor coordinate frame. In (6) shown above, the cost function to be minimized is the sum-of-squared error between the magnetic field prediction from (1) and the measurements.

A number of algorithms have been used to solve this nonlinear optimization problem, such as the steepest gradient descent algorithm and Newton's method. Exemplary embodiments of the present disclosure utilize the Levenberg-Marquardt (L-M) algorithm for solving (6), which is suitable for solving nonlinear least-square problems in real-time [75]. The L-M algorithm utilizes the Jacobian matrix at the current estimation to perform parameter updates in a descent direction iteratively toward a local minimum. A common way to calculate the Jacobian matrix is numerically computing the derivatives by evaluating the prediction error at least once for each parameter, which is computationally expensive and unsuitable for real-time magnetic location estimation. Aiming to achieve minimum latency to allow for real-time localization while attaining acceptable estimation accuracy, an analytical expression of the Jacobian matrix is computed by taking derivatives of the magnetic field prediction in sensor frame (5) with respect to the parameters as

$${J}_{i} = {T}_{i} \frac{\partial {B}_{i}}{\partial ξ} , \qquad ( 7 )$$

where Jᵢ ∈R^{3×5} is the Jacobian submatrix corresponding to i-th sensor. The total Jacobian matrix is composed of all Jacobian submatrices Jᵢ across Ns sensors. The detailed expression of the analytical-from Jᵢ is presented as the first 5 columns of (11) in [71].

A proper initial estimate must be used to start the iterations during solving (6) to guarantee fast convergence. Assuming the catheter motion is sufficiently slow and thus the magnet's position variation during the computational latency is small, a warm start strategy [32] is used in this work, where the optimal estimation of the current estimation is used as the initial condition of the next time step. Such an initialization strategy can facilitate the convergence of the L-M iteration and reduce the number of iterations required for convergence during real-time localization.

### Calibration Algorithm

### Ambient Field Removal

In particular embodiments of MSRC systems disclosed herein, the magnetic field from the source magnet at the sensor locations is below 8 Gauss, which is in the same order of magnitude as the earth's field. The ambient field signal needs to be removed from the sensor measurements to provide estimation accuracy. To achieve this, the inventors first record the sensors' reading for earth's magnetic field without a magnet in the measurement range. This ambient field signal is then subtracted from the sensor readings in all sensor data to get the measurement of the field from the magnet. Note that the earth field measurement needs to be repeated if the system is being moved or rotated to maintain accurate magnetic localization.

### Sensor Position and Magnetic Moment Calibration

In the magnetic localization approach shown in (6), the positions for all magnetic sensors psi and the strength M of the PM to be localized are assumed to be known a priori.

However, the actual sensor locations can deviate from their nominal locations due to inaccuracy in the mechanical design and assembly, and the discrepancy between the PM's strength and its nominal material properties. To address these issues, sensor data should be collected when the magnet is located at a series of known positions and orientations for calibration. One calibration approach introduced in uses a sequential approach to estimate the magnet's dipole moment M and i-th sensor location psi as

$$\hat{M} = {arg min}_{M} {\sum }_{j = 1}^{{N}_{d}} {\sum }_{i = 1}^{{N}_{s}} {‖ M {s}^{} {\overline{B}}_{ij} - {s}^{} {B}_{ij}^{′} ‖}^{2} , \qquad ( 8 )$$

$${\hat{p}}_{si} = {arg min}_{{p}_{i}} {\sum }_{j = 1}^{{N}_{d}} {‖ {s}^{} {B}_{ij} ( {p}_{si} , M , {p}_{mj} , {θ}_{j} , {ϕ}_{j} ) - {s}^{} {B}_{ij}^{′} ‖}^{2} , \qquad ( 9 )$$

where N_{d} is the number of all calibration data points, ^{s}B′ᵢⱼ, is the i-th sensor measurement for j-th calibration data, (pₘⱼ, 0ⱼ, ϕⱼ) are the position and orientation angles for the PM when taking the j-th data.

This problem solves for the optimal sensor locations that minimize the error between the estimated and true magnet locations. The nominal sensor locations are used to initiate the L-M iterations. Note that the optimal solution for dipole moment \hat{M} obtained from (8) is used when solving for the sensor location estimation problem (9). This approach has demonstrated improvements in localization accuracy in [69]; however, due to the strong inter-dependency between the two optimization problems, a sequential calibration method in (9). This approach has demonstrated improvements in localization accuracy in [69]; however, due to the strong inter-dependency between the two optimization problems, a sequential calibration method in (8)-(9) can lead to sub-optimal calibration results. To remove the coupling between the two problems and achieve the optimal calibration results, ideally, the sensor location and magnetic dipole moment should be estimated simultaneously as formulated below:

$${min}_{M , {p}_{si} , ∀ i} E = {\sum }_{j = 1}^{{N}_{d}} {\sum }_{i = 1}^{{N}_{s}} {‖ M {}^{s} {\overline{B}}_{ij} ( {p}_{si} ) - {s}^{} {B}_{ij}^{′} ‖}^{2} , \qquad ( 10 )$$

This optimization problem, however, is highly nonlinear and requires a large and highly-sparse Jacobian matrix with a dimension of (NₛN_{d})×(3Nₛ+1), which is computationally expensive and is impractical for sensor calibration.

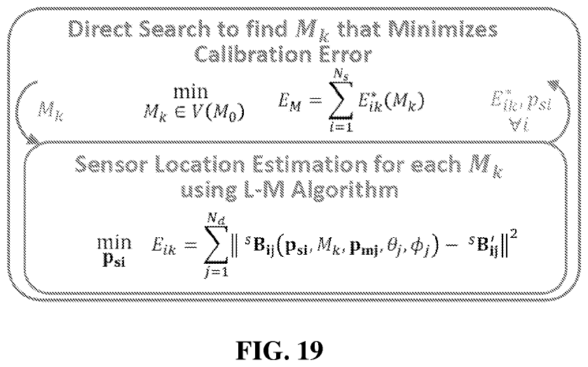

Aiming to attain the overall optimal calibration results in sensor location and magnetic moment while maintaining computational simplicity, a new calibration approach is proposed in this paper as shown in FIG. 19. This proposed approach takes a nested framework to estimate the optimal magnetic dipole strength M and sensor locations pₛᵢ. For each magnetic dipole moment value Mₖ in a feasible range V(M₀) (around the vicinity of the nominal dipole strength M₀), the sensor location estimation problem (9) is solved for each Mₖ. The optimal magnetic dipole moment magnetic dipole moment \hat{M} can then be found through a direct search approach, which produces the minimum localization error over all sensors, and the optimal sensor positions p\hat{ }ₛᵢ can be found from the optimum in (9) with \hat{M} being used. To speed up the computation, a coarse search can first be used to narrow the searching range, and a fine search can then be conducted to find the optimal M with sufficient accuracy. Compared with sequential estimation formulation in [69] and a simultaneous estimation formulation in (10), the proposed nested calibration approach (i) effectively addresses the coupling between two optimization problems, (ii) is computationally efficient, and (iii) can guarantee optimality [77]. The performance of the proposed new calibration approach is presented in section below entitled “Results and Discussion.”

### Integrating Actuation and Localization

This section discusses the integration of the magnetic localization algorithm described in the previous section entitled “Realtime Localization Algorithm”, and the magnetic steering for MSRC systems for PEI procedures”. Due to the diameter constraint of catheters, the magnet at the catheter tip should have a diameter of 3 mm or smaller [60], and the magnetic sensors being used for the localization should be highly sensitive to capture the magnetic field from the catheter tip. However, such highly-sensitive magnetic sensors (e.g. magneto-resistive sensors for earth field measurement) typically have a relatively low measurement range. One example is the high-resolution magneto-resistive sensor QMC5883L, which has a high resolution of 5 mGauss, but its measurement range is +8 Gauss. The magnetic actuation in MSRC systems typically exceeds 100 Gauss to steer the catheter tip, which will saturate the sensors and thus disable the magnetic localization. Hence, an integration method needs to be created to enable the smooth switching between magnetic steering (under x-ray fluoroscopic imaging guidance) and magnetic catheter tip tracking while minimizing x-ray radiation required to ensure safety during catheter navigation.

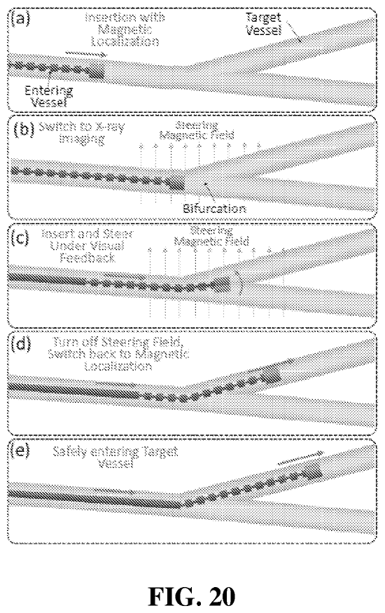

FIG. 20 shows a schematic diagram of one exemplary embodiment of a robotic catheter as it navigates through a bifurcation in the vascular structure under integrated magnetic steering and localization under the assistance of x-ray imaging only during steering. A radiographic (e.g. x-ray) image of the vascular structure is acquired before PEI therapy. During the PEI procedure, as the catheter is advanced into a non-bifurcated (e.g. straight) segment of the vessel as shown in step (a), magnetic localization is used to provide real-time feedback on the catheter tip location to the surgeon, and no steering field is needed. When the catheter tip comes to the proximity of the bifurcated portion of the vascular structure, the localization method for the catheter switches to a radiographic (e.g. x-ray imaging), and a magnetic field is turned on to steer the robotic catheter towards the target branch, as shown in steps (b)-(c). Once the catheter tip successfully selects and is inserted in the proximal segment of the target branch, the steering field can then be turned off, and the localization of the catheter is switched back to magnetic localization, which guides further advancement of the robotic catheter towards the target lesion as shown in steps (d)-(e). A smooth transition between the magnetic steering (under x-ray imaging) and magnetic localization should be implemented to avoid sudden changes in the bending force for the catheter tip. Through this proposed procedure, fluoroscopic imaging in the navigation of an MSRC is significantly reduced to only bifurcation catheterization, which is highly effective in reducing the x-ray exposure to both the patient and surgeon during interventions.

### Experimental Setup

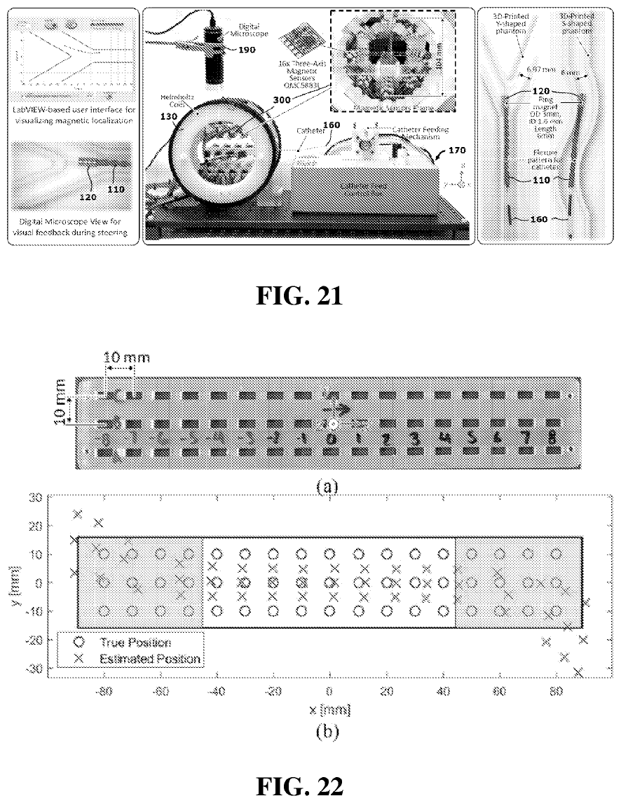

FIG. 21 shows an overview of an MSRC prototype system integrating magnetic steering and localization according to exemplary embodiments of the present disclosure. This embodiment comprises a catheter 160 with a flexible segmented tubular member 110 and a permanent magnet 120, a steering actuator using electromagnet (e.g. Helmholtz coils) 130, a catheter feeding mechanism 180, a magnetic sensor array 300 for catheter tip localization, a digital microscope simulating the fluoroscopic imaging in PEI processes, and a 3D-printed vasculature phantom. The hardware design of the MSRC prototype (except for the magnetic localization system comprising magnetic sensor array 300) is similar to embodiments previously described herein.

As previously described, the catheter in this specific embodiment comprises a commercially available catheter (G11430, Cook Medical, USA) and a flexible distal end made of a heat shrink tube (40142000, MILAPEAK) with 3.2 mm pre-shrinking outer diameter and 40 mm length. Flexure patterns are made on the catheter distal end to reduce its stiffness and thus increase steerability. The modeling, design optimization, and fabrication procedures for the catheter distal end are presented in [61] and [60]. A stack of ring-shaped magnets N52 grade NdFeB, K&J Magnetics) with 3 mm outer diameter, 1.6 mm inner diameter, and a length of 6 mm is coupled to the tip of the catheter to allow for magnetic steering and localization, as is shown in FIG. 21. The magnetic steering actuator being used is a pair of custom-fabricated Helmholtz coils aiming to create a uniform magnetic field for steering of the MSRC. In the illustrated embodiment, the Helmholtz coils have a diameter of 220 mm and are configured with a distance of 110 mm. The coils are connected in series and are driven by full-bridge bi-directional switching-type power amplifiers (e.g. B07NP6XNPR, Cytron Technologies©) with 20 A current capacity. In this embodiment, the coils have 120 turns each under the maximum allowed current amplitude of 11 A (limited by the power supply being selected). The insertion of the catheter uses a feeding mechanism comprised of a friction drive driven by a stepper motor. The design details for the catheter feeding mechanism are equivalent to those in embodiments previously described herein.

In addition to the MSRC prototype system described above, a cylindrical array of magnetic sensors 300 is arranged surrounding the catheter tip for localization, as is shown in FIG. 21. A high-resolution magneto-resistive sensor QMC5883L (5 mGauss resolution, measurement range ±8 Gauss) is selected for the localization. In the embodiment shown, a total of 32 sensors are used for the cylindrical sensor array, and the y-directional distance between two sensors is 25 mm. The diameter of the cylindrical sensor array is 104 mm. In this embodiment, only 16 of the sensors (top, bottom, left, and right columns) are used for magnetic localization to allow for faster instrumentation and computation. Over-sampling is used for all sensor signals for noise reduction. In the embodiment shown, the sensor signals are collected into a myRIO real-time controller (National Instruments®) through I2C interface via two 8-to-1 multiplexers and the latency for data collection is 5 ms. In this embodiment, the real-time magnetic localization and actuation algorithms discussed in Sections entitled “Realtime Localization Algorithm” and “Integrating Actuation and Localization”, are implemented in the myRIO real-time controller with a computation latency of approximately 400 ms, which is sufficiently fast for real-time feedback for the human operator.

### Results and Discussion

### Magnetic Localization and Calibration Tests

To evaluate the localization performance and to calibrate the system, two different 3D-printed calibration grids are fabricated to place the PM being localized at different defined locations. In view FIG. 22 view (a) a photograph for the 3D-printed calibration plate I to place the magnet at varying defined locations is shown. FIG. 22 view (b) shows a comparison between the true and estimated magnet locations using calibration plate I without sensor location calibration. The average translational localization error over the entire plate is 10.8 mm, and that of the middle section is 4.5 mm The magnetic sensor readings were first collected with the magnet placed at all locations in the calibration plate shown in FIG. 22 view (a) and conducted magnetic localization using the measured data. FIG. 22 view (b) shows the magnetic localization performance assuming the sensors are at their nominal locations. It can be observed that the magnet position estimation error is small when the magnet is within the center section of the plate, i.e., x∈[−40 mm, 40 mm], and can reach 20 mm error when the magnet is at the edge of the calibration plate. This is because the magnet is out of the cylindrical sensor array when it is located at the front and back ends of the calibration plate, reducing the signal-to-noise ratio of sensor readings. To obtain high-quality signals and thus provide accurate localization performance, the sensor array needs to cover the magnet location.

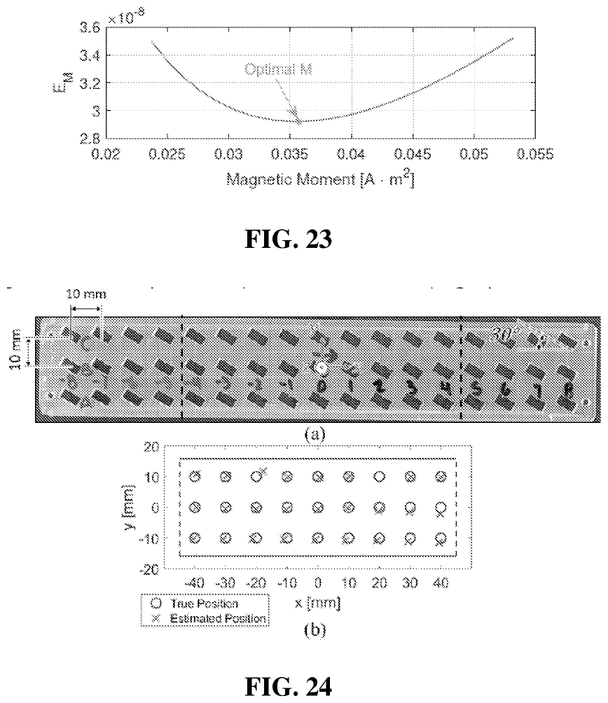

To improve the localization accuracy, the calibration for sensor location and magnetic dipole moment strength using the algorithm introduced in FIG. 19 was performed for the exemplary embodiment. The data shown in FIG. 22 is used to perform the calibration, and FIG. 23 shows the variation of the objective function in FIG. 19 as the value of magnet's dipole moment M varies, demonstrating the convergence of the direct search approach in the proposed nested calibration formulation. The computation time for the calibration problem solving is approximately 20 minutes on a regular desktop computer.

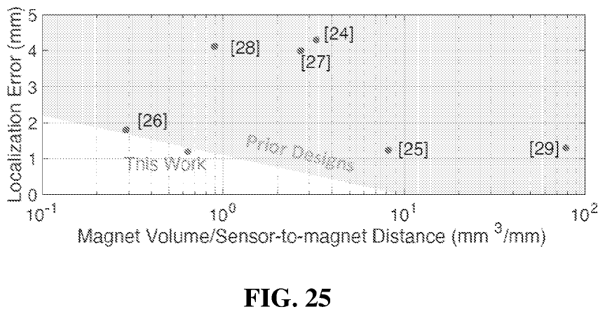

To validate the performance of the proposed sensor calibration procedure, a new calibration plate II shown in FIG. 24 view (a) was used. Localization was performed with the magnet placed at each location in the calibration plate II with and without the calibrations. FIG. 24 view (b) shows the magnetic localization result after the calibrations. Table I below summarizes the average translation and angular errors before and after calibration, where the localization was conducted for the magnet at 51 locations (all data). It can be seen that the magnetic localization error in the center range (i.e., the region within the cylindrical sensor array, 27 points) is improved from 4.34 mm to 1.27 mm, making it sufficiently accurate for the considered MSRC application. Moreover, FIG. 25 illustrates the magnetic localization performance comparison between embodiments of the present disclosure and the prior studies, in which Vₘ/|pᵢ| is selected as the metric for the horizontal axis since the field from a magnetic dipole is proportional to this value. Thanks to the highly effective sensor calibration approach, embodiments of the present disclosure demonstrate excellent overall performance in terms of accuracy and Vₘ/|pᵢ| compared to the previous literature.

TABLE I
AVERAGE LOCALIZATION ERROR WITH
AND WITHOUT CALIBRATIONS

| Data Range | w/o calibration | | w/calibration |
|---|---|---|---|
| All data (51 pts) translation error | 10.16 | mm | 4.53 mm |
| All data (51 pts) angular error | 6.07°/12.48° | | 3.77°/8.62° |
| Center range (27 pts) translation error | 4.34 | mm | 1.27 mm |

| | | |
|---|---|---|
| Center range (27 pts) angular error | 1.46°/3.84° | 0.98°/1.82° |

### MSRC Navigation Experiments

To demonstrate the effectiveness of the proposed integration of magnetic localization and magnetic steering in MSRC systems, experiments of navigating the catheter over 3D-printed phantoms of lower extremity vascular structure are conducted. The proposed switching procedure between magnetic location sensing and magnetic steering introduced in section entitled “Integrating Actuation and Localization” is used at bifurcation locations. During these case studies, a human operator can control the insertion and switch between magnetic steering and magnetic localization. Of note, in exemplary embodiments of the present disclosure, the proposed magnetic localization was solely used to provide visual feedback to the user during the insertion procedure to minimize exposure to x-ray radiations, while the digital microscope video (simulating x-ray imaging) was used as the visual feedback during the magnetic steering step.

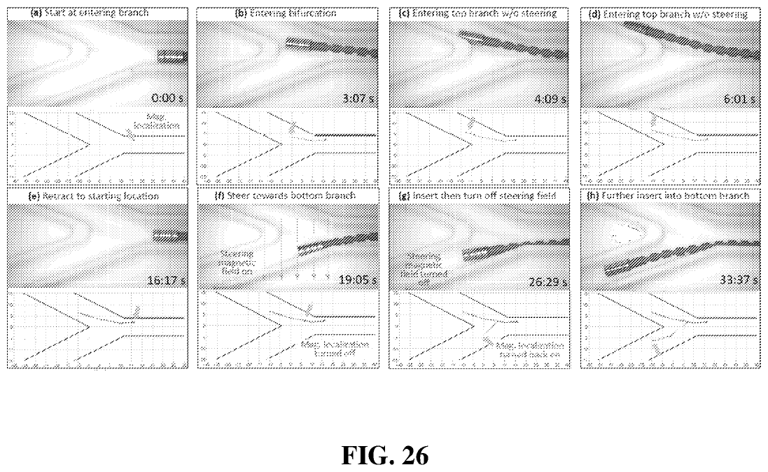

FIG. 26 shows the snapshots of the microscope video and the real-time user interface screenshot of the MSRC navigating through a Y-shaped phantom of the vasculature. In this case study, the top branch of the Y-shaped phantom is the natural branch that the catheter gets in without magnetic steering due to the pre-bending of the catheter tip, and steering is required for the catheter to get into the bottom branch, simulating a situation during a realistic PEI in which catheter tends to enter to an unwanted branch of a bifurcation. In this test, the catheter was first inserted into the top branch in the Y-shaped phan-tom with the operator under visual feedback using magnetic location sensing as shown in FIG. 26, views (a)-(d). Then, the catheter was retracted to the starting location as shown in FIG. 26 view (e) and switched to magnetic steering using the Helmholtz coils. In this step, the operator can control the steering field and catheter advancement under visual feedback from the digital microscope video (or under x-ray guidance) as shown in FIG. 26 view (f). After the catheter enters the target branch, the operator can turn off the steering field and switch back to magnetic localization for catheter tip location sensing, as shown in FIG. 26 view (g). Next, the operator can insert the catheter into the target branch under feedback using magnetic localization. The successful navigation of the catheter into both target branches in the phantom validates the effectiveness of the magnetic location sensing and steering approach according to exemplary embodiments of the present disclosure.

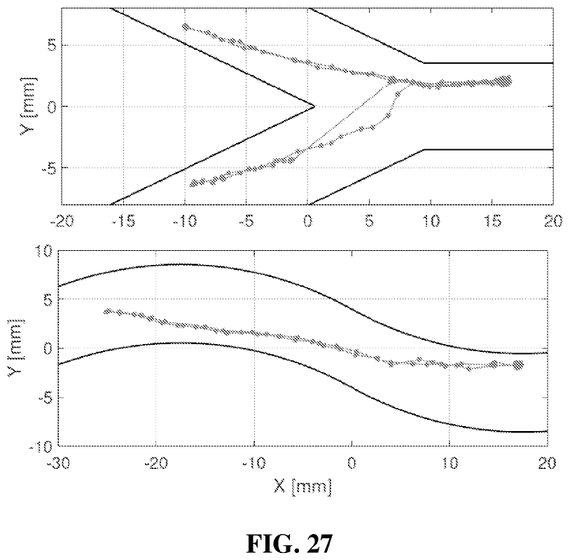

The catheter navigation test was repeated for the S-shaped phantom, which simulates the varying curvatures in human vasculatures. Since there are no bifurcations in the S-shaped phantom, magnetic location sensing was used throughout the navigation process, and no magnetic steering or digital microscope imaging feedback was required. FIG. 27 shows the magnetic location sensing results of the catheter tip tested with both phantoms. During these tests, the operator was able to successfully control the feed of the catheter while only looking at the user interface showing the magnetic localization result, and only needed to look at the digital microscope image during steering. These tests validate the effectiveness of the method integrating magnetic steering and magnetic location sensing for MSRCs to minimize x-ray radiation for catheter navigation disclosed herein.

### CONCLUSIONS

Exemplary embodiments of the present disclosure include a new magnetic location sensing and actuation approach for magnetically steerable robotic catheters, aiming to reduce the x-ray exposure to the surgeon and the patient during intervention surgeries. The real-time magnetic location sensing uses the L-M algorithm to solve a least square estimation problem with 400 ms latency, which is sufficiently fast for visual feedback for the human operator during catheter insertion. Exemplary embodiments also include a new nested calibration framework for sensor locations and the dipole moment strength of the magnet, which addresses the coupling challenge in prior introduced sequential calibration approaches. To address the challenge that the actuation field can saturate location sensing sensors, a novel integration approach is proposed for magnetic steering and magnetic location sensing, which reduce the need for x-ray usage to only at bifurcation locations. Experiments show that the magnetic location sensing error for a ring-shaped permanent magnet (3 mm OD, 1.6 mm ID, 6 mm length) over the sensing region is 1.27 mm, which well satisfies the required sensing accuracy. Catheter navigation tests verify the effectiveness of the proposed approach to integrating magnetic location sensing and magnetic steering for MSRCs.

All of the devices, systems and/or methods disclosed and claimed herein can be made and executed without undue experimentation in light of the present disclosure. While the devices, systems and methods of this invention have been described in terms of particular embodiments, it will be apparent to those of skill in the art that variations may be applied to the devices, systems and/or methods in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

The contents of the following references are incorporated by reference herein:
[1] G. A. Roth, C. Johnson, A. Abajobir, F. Abd-Allah, S. F. Abera, G. Abyu, M. Ahmed, B. Aksut, T. Alam, K. Alam, et al., “Global, regional, and national burden of cardiovascular diseases for 10 causes, 1990 to 2015,” Journal of the American College of Cardiology, vol. 70, no. 1, pp. 1-25, 2017.
[2] F. G. R. Fowkes, D. Rudan, I. Rudan, V. Aboyans, J. O. Denenberg, M. M. McDermott, P. E. Norman, U. K. Sampson, L. J. Williams, G. A. Mensah, et al., “Comparison of global estimates of prevalence and risk factors for peripheral artery disease in 2000 and 2010: a systematic review and analysis,” The lancet, vol. 382, no. 9901, pp. 1329-1340, 2013.
[3] U. Campia, M. Gerhard-Herman, G. Piazza, and S. Z. Goldhaber, “Peripheral artery disease: past, present, and future,” The American journal of medicine, vol. 132, no. 10, pp. 1133-1141, 2019.
[4] M. H. Shishehbor and M. R. Jaff, “Percutaneous therapies for pe-ripheral artery disease,” Circulation, vol. 134, no. 24, pp. 2008-2027, 2016.
[5] M. Goyal, A. Y. Yu, B. K. Menon, D. W. Dippel, W. Hacke, S. M. Davis, M. Fisher, D. R. Yavagal, F. Turjman, J. Ross, et al., “Endovas-cular therapy in acute ischemic stroke: challenges and transition from trials to bedside,” Stroke, vol. 47, no. 2, pp. 548-553, 2016.
[6] “SwiftNINJA Steerable Microcatheter,” https://www.merit.com/peripheral-intervention/delivery-systems/microcatheters/swiftninja-steerable-microcatheter, accessed: September 2021.
[7] S. Jeon, A. K. Hoshiar, S. Kim, S. Lee, E. Kim, S. Lee, K. Kim, J. Lee, J.-y. Kim, and H. Choi, “Improving guidewire-mediated steerability of a magnetically actuated flexible microrobot,” Micro and Nano Systems Letters, vol. 6, no. 1, pp. 1-10, 2018.
[8] S. Jeon, A. K. Hoshiar, K. Kim, S. Lee, E. Kim, S. Lee, J.-y. Kim, B. J. Nelson, H.-J. Cha, B.-J. Yi, et al., “A magnetically controlled soft microrobot steering a guidewire in a three-dimensional phantom vascular network,” Soft robotics, vol. 6, no. 1, pp. 54-68, 2019.
[9] N. Kim, S. Lee, W. Lee, and G. Jang, “Development of a magnetic catheter with rotating multi-magnets to achieve unclogging motions with enhanced steering capability,” AIP Advances, vol. 8, no. 5, p. 056708, 2018.
[10] L. Wang, D. Zheng, P. Harker, A. B. Patel, C. F. Guo, and X. Zhao, “Evolutionary design of magnetic soft continuum robots,” Proceedings of the National Academy of Sciences, vol. 118, no. 21, 2021.
[11] J. Hwang, J.-y. Kim, and H. Choi, “A review of magnetic actua-tion systems and magnetically actuated guidewire-and catheter-based microrobots for vascular interventions,” Intelligent Service Robotics, vol. 13, no. 1, pp. 1-14, 2020.
[12] A. D. Losey, P. Lillaney, A. J. Martin, D. L. Cooke, M. W. Wilson,
B. R. Thorne, R. S. Sincic, R. L. Arenson, M. Saeed, and S. W. Hetts, “Magnetically assisted remote-controlled endovascular catheter for interventional mr imaging: in vitro navigation at 1.5 t versus x-ray fluoroscopy,” Radiology, vol. 271, no. 3, pp. 862-869, 2014.
[13] T. L. Thomas, V. K. Venkiteswaran, G. Ananthasuresh, and S. Misra, “A monolithic compliant continuum manipulator: a proof-of-concept study,” Journal of mechanisms and robotics, vol. 12, no. 6, p. 061006, 2020.
[14] V. N. Le, N. H. Nguyen, K. Alameh, R. Weerasooriya, and P. Pratten, “Accurate modeling and positioning of a magnetically controlled catheter tip,” Medical physics, vol. 43, no. 2, pp. 650-663, 2016.
[15] C. Chautems, A. Tonazzini, Q. Boehler, S. H. Jeong, D. Floreano, and B. J. Nelson, “Magnetic continuum device with variable stiffness for minimally invasive surgery,” Advanced Intelligent Systems, vol. 2, no. 6, p. 1900086, 2020.
[16] Y. Kim, G. A. Parada, S. Liu, and X. Zhao, “Ferromagnetic soft continuum robots,” Science Robotics, vol. 4, no. 33, p. eaax7329, 2019.
[17] B. E. Kratochvil, M. P. Kummer, S. Erni, R. Borer, D. R. Frutiger,
S. Schu\ddot{ }rle, and B. J. Nelson, “Minimag: a hemispherical electromag-netic system for 5-dof wireless micromanipulation,” in Experimental Robotics. Springer, 2014, pp. 317-329.
[18] R. Lorbeer, A. Grotz, M. Do\hat{ }rr, H. Vo\hat{ }lzke, W. Lieb, J.-P. Ku\hat{ }hn, and
B. Mensel, “Reference values of vessel diameters, stenosis prevalence, and arterial variations of the lower limb arteries in a male population sample using contrast-enhanced mr angiography,” PloS one, vol. 13, no. 6, p. e0197559, 2018.
[19] R. J. Murphy, M. D. Kutzer, S. M. Segreti, B. C. Lucas, and
M. Armand, “Design and kinematic characterization of a surgical manipulator with a focus on treating osteolysis,” Robotica, vol. 32, no. 6, pp. 835-850, 2014.
[20] F. Alambeigi, Y. Wang, S. Sefati, C. Gao, R. J. Murphy, I. Iordachita,
R. H. Taylor, H. Khanuja, and M. Armand, “A curved-drilling ap-proach in core decompression of the femoral head osteonecrosis using a continuum manipulator,” IEEE Robotics and Automation Letters, vol. 2, no. 3, pp. 1480-1487, 2017.
[21] H. A. Haus and J. R. Melcher, Electromagnetic fields and energy. Prentice Hall Englewood Cliffs, NJ, 1989, vol. 107.
[22] J. Wang, S. She, and S. Zhang, “An improved helmholtz coil and analysis of its magnetic field homogeneity,” Review of scientific instruments, vol. 73, no. 5, pp. 2175-2179, 2002.
[23] T. Moix, D. Ilic, B. Fracheboud, and H. Bleuler, “Design of a friction drive actuator with integrated force and torque sensors,” in 2005 IEEE Instrumentation and Measurement Technology Conference Proceedings, vol. 3. IEEE, 2005, pp. 1762-1766.
[24] M. Noh, D. P. Mooney, and D. L. Trumper, “Magnet-assisted hydraulic bougienage for correction of long-gap esophageal atresia,” IEEE Transactions on Biomedical Engineering, vol. 65, no. 10, pp. 2178-2189, 2017.
[25] F. Alambeigi, S. A. Pedram, J. L. Speyer, J. Rosen, I. Iordachita,
R. H. Taylor, and M. Armand, “Scade: Simultaneous sensor calibration and deformation estimation of fbg-equipped unmodeled continuum manipulators,” IEEE Transactions on Robotics, vol. 36, no. 1, pp. 222-239, 2019.
[26] M. D. Gerhard-Herman, H. L. Gornik, C. Barrett, N. R. Barshes, M. A. Corriere, D. E. Drachman, L. A. Fleisher, F. G. R. Fowkes, N. M. Hamburg, S. Kinlay et al., “2016 aha/acc guideline on the management of patients with lower extremity peripheral artery disease: a report of the american college of cardiology/american heart association task force on clinical practice guidelines,” Journal of the American College of Cardiology, vol. 69, no. 11, pp. e71-e126, 2017.
[27] M. H. Shishehbor and M. R. Jaff, “Percutaneous therapies for peripheral artery disease,” Circulation, vol. 134, no. 24, pp. 2008-2027, 2016.
[28] J. Hwang, J.-y. Kim, and H. Choi, “A review of magnetic actuation systems and magnetically actuated guidewire-and catheter-based micro-robots for vascular interventions,” Intelligent Service Robotics, vol. 13, no. 1, pp. 1-14, 2020.
[29] L. Wang, D. Zheng, P. Harker, A. B. Patel, C. F. Guo, and X. Zhao, “Evolutionary design of magnetic soft continuum robots,” Proceedings of the National Academy of Sciences, vol. 118, no. 21, 2021.
[30] L. B. Kratchman, T. L. Bruns, J. J. Abbott, and R. J. Webster, “Guiding elastic rods with a robot-manipulated magnet for medical applications,” IEEE Transactions on Robotics, vol. 33, no. 1, pp. 227-233, 2016.
[31] J. Edelmann, A. J. Petruska, and B. J. Nelson, “Magnetic control of continuum devices,” The International Journal of Robotics Research, vol. 36, no. 1, pp. 68-85, 2017.
[32] F. Carpi and C. Pappone, “Stereotaxis Niobe® magnetic navigation system for endocardial catheter ablation and gastrointestinal capsule endoscopy,” Expert review of medical devices, vol. 6, no. 5, pp. 487-498, 2009.
[33] B. L. Nguyen, J. L. Merino, and E. S. Gang, “Remote navigation for ablation procedures-a new step forward in the treatment of cardiac arrhythmias,” European Cardiology, vol. 6, no. 3, pp. 50-56, 2010.
[34] V. Lalande, F. P. Gosselin, M. Vonthron, B. Conan, C. Tremblay,
G. Beaudoin, G. Soulez, and S. Martel, “In vivo demonstration of magnetic guidewire steerability in a mri system with additional gradient coils,” Medical physics, vol. 42, no. 2, pp. 969-976, 2015.
[35] T. L. Thomas, V. K. Venkiteswaran, G. Ananthasuresh, and S. Misra, “A monolithic compliant continuum manipulator: a proof-of-concept study,” Journal of mechanisms and robotics, vol. 12, no. 6, p. 061006, 2020.
[36] S. S. Antman, “Nonlinear problems of elasticity,” Springer, 1995.
[37] D. C. Rucker and R. J. Webster III, “Statics and dynamics of contin-uum robots with general tendon routing and external loading,” IEEE Transactions on Robotics, vol. 27, no. 6, pp. 1033-1044, 2011.
[38] J. Till, V. Aloi, and C. Rucker, “Real-time dynamics of soft and continuum robots based on cosserat rod models,” The International Journal of Robotics Research, vol. 38, no. 6, pp. 723-746, 2019.
[39] C. B. Black, J. Till, and D. C. Rucker, “Parallel continuum robots: Mod-eling, analysis, and actuation-based force sensing,” IEEE Transactions on Robotics, vol. 34, no. 1, pp. 29-47, 2017.
[40] H. A. Haus and J. R. Melcher, Electromagnetic fields and energy. Prentice Hall Englewood Cliffs, NJ, 1989, vol. 107.
[41] I. S. Grant and W. R. Phillips, Electromagnetism. John Wiley & Sons, 2013.
[42] J. Wang, S. She, and S. Zhang, “An improved helmholtz coil and analysis of its magnetic field homogeneity,” Review of scientific instruments, vol. 73, no. 5, pp. 2175-2179, 2002.
[43] Y. Liu and F. Alambeigi, “Effect of external and internal loads on tension loss of tendon-driven continuum manipulators,” IEEE Robotics and Automation Letters, vol. 6, no. 2, pp. 1606-1613, 2021.
[44] C. T. Kelley, Iterative methods for linear and nonlinear equations. SIAM, 1995.
[45] “Centers for disease control and prevision: Peripheral arterial disease (PAD),” https://www.cdc.gov/heartdisease/PAD.htm, accessed: Feb. 19, 2023.
[46] U. Campia, M. Gerhard-Herman, G. Piazza, and S. Z. Goldhaber, “Peripheral artery disease: past, present, and future,” The American journal of medicine, vol. 132, no. 10, pp. 1133-1141, 2019.
[47] M. H. Shishehbor and M. R. Jaff, “Percutaneous therapies for peripheral artery disease,” Circulation, vol. 134, no. 24, pp. 2008-2027, 2016.
[48] M. Goyal, A. Y. Yu, B. K. Menon, D. W. Dippel, W. Hacke, S. M. Davis,
M. Fisher, D. R. Yavagal, F. Turjman, J. Ross, et al., “Endovascular therapy in acute ischemic stroke: challenges and transition from trials to bedside,” Stroke, vol. 47, no. 2, pp. 548-553, 2016.
[49] “SwiftNINJA Steerable Microcatheter,” https://www.merit.com/peripheral-intervention/delivery-systems/microcatheters/swiftninja-steerable-microcatheter, accessed: September 2021.
[50] M. Khoshnam and R. V. Patel, “Robotics-assisted control of steerable ablation catheters based on the analysis of tendon-sheath transmission mechanisms,” IEEE/ASME Transactions on Mechatronics, vol. 22, no. 3,
pp. 1473-1484, 2017.
[51] X. Hu, A. Chen, Y. Luo, C. Zhang, and E. Zhang, “Steerable catheters for minimally invasive surgery: a review and future directions,” Computer Assisted Surgery, vol. 23, no. 1, pp. 21-41, 2018.
[52] L. Wang, D. Zheng, P. Harker, A. B. Patel, C. F. Guo, and X. Zhao, “Evolutionary design of magnetic soft continuum robots,” Proc. Natl. Acad. Sci. U.S.A., vol. 118, no. 21, p. e2021922118, 2021.
[53] L. B. Kratchman, T. L. Bruns, J. J. Abbott, and R. J. Webster, “Guiding elastic rods with a robot-manipulated magnet for medical applications,” IEEE Transactions on Robotics, vol. 33, no. 1, pp. 227-233, 2016.
[54] J. Edelmann, A. J. Petruska, and B. J. Nelson, “Magnetic control of continuum devices,” The International Journal of Robotics Research, vol. 36, no. 1, pp. 68-85, 2017.
[55] F. Carpi and C. Pappone, “Stereotaxis Niobe® magnetic navigation system for endocardial catheter ablation and gastrointestinal capsule endoscopy,” Exp. rev. of med. devices, vol. 6, no. 5, pp. 487-498, 2009.
[56] B. L. Nguyen, J. L. Merino, and E. S. Gang, “Remote navigation for ablation procedures-a new step forward in the treatment of cardiac arrhythmias,” European Cardiology, vol. 6, no. 3, pp. 50-56, 2010.
[57] J. Hwang, J.-y. Kim, and H. Choi, “A review of magnetic actuation systems and magnetically actuated guidewire-and catheter-based micro-robots for vascular interventions,” Intelligent Service Robotics, vol. 13, no. 1, pp. 1-14, 2020.
[58] L. Wang, C. F. Guo, and X. Zhao, “Magnetic soft continuum robots with contact forces,” Extreme Mechanics Letters, vol. 51, p. 101604, 2022.
[59] C. Heunis, J. Sikorski, and S. Misra, “Flexible instruments for endovas-cular interventions: Improved magnetic steering, actuation, and image-guided surgical instruments,” IEEE robotics & automation magazine, vol. 25, no. 3, pp. 71-82, 2018.
[60] T. G. Mohanraj, J. Song, M. R. Rajebi, L. Zhou, and F. Alambeigi, “A kirigami-based magnetically steerable robotic catheter for treatment of peripheral artery disease,” in 2022 9th IEEE RAS/EMBS International Conference for Biomedical Robotics and Biomechatronics (BioRob). IEEE, 2022, pp. 1-6.
[61] Y. Liu, T. G. Mohanraj, M. R. Rajebi, L. Zhou, and F. Alambeigi, “Mul-tiphysical analytical modeling and design of a magnetically steerable robotic catheter for treatment of peripheral artery disease,” IEEE/ASME Transactions on Mechatronics, vol. 27, no. 4, pp. 1873-1881, 2022.
[62] T.-H. Chiang, Z.-H. Sun, H.-R. Shiu, K. C.-J. Lin, and Y.-C. Tseng, “Magnetic field-based localization in factories using neural network with robotic sampling,” IEEE Sensors Journal, vol. 20, no. 21, pp. 13 110-13 118, 2020.
[63] C. Hu, M. Q. Meng, and M. Mandal, “Efficient magnetic localization and orientation technique for capsule endoscopy,” in 2005 IEEE/RSJ International Conference on Intelligent Robots and Systems. IEEE, 2005, pp. 628-633.
[64] C. Di Natali, M. Beccani, N. Simaan, and P. Valdastri, “Jacobian-based iterative method for magnetic localization in robotic capsule endoscopy,” IEEE Transactions on Robotics, vol. 32, no. 2, pp. 327-338, 2016.
[65] Q. Fu, C. Fan, X. Wang, S. Zhang, X. Zhang, J. Guo, and S. Guo, “A compensation method for magnetic localization on capsule robot in medical application,” IEEE Sensors Journal, vol. 21, no. 23, pp. 26 690-26 698, 2021.
[66] S. Guitron, A. Guha, S. Li, and D. Rus, “Autonomous locomotion of a miniature, untethered origami robot using hall effect sensor-based magnetic localization,” in 2017 IEEE International Conference on Robotics and Automation (ICRA). IEEE, 2017, pp. 4807-4813.
[67] D. Son, X. Dong, and M. Sitti, “A simultaneous calibration method for magnetic robot localization and actuation systems,” IEEE Transactions on Robotics, vol. 35, no. 2, pp. 343-352, 2018.
[68] C. Watson and T. K. Morimoto, “Permanent magnet-based localization for growing robots in medical applications,” IEEE Robotics and Au-tomation Letters, vol. 5, no. 2, pp. 2666-2673, 2020.
[69] S. Su, W. Yang, H. Dai, X. Xia, M. Lin, B. Sun, and C. Hu, “Investiga-tion of the relationship between tracking accuracy and tracking distance of a novel magnetic tracking system,” IEEE Sensors Journal, vol. 17, no. 15, pp. 4928-4937, 2017.
[70] N. Sebkhi, N. Sahadat, S. Hersek, A. Bhavsar, S. Siahpoushan,
M. Ghoovanloo, and O. T. Inan, “A deep neural network-based perma-nent magnet localization for tongue tracking,” IEEE Sensors Journal, vol. 19, no. 20, pp. 9324-9331, 2019.
[71] C. R. Taylor, H. G. Abramson, and H. M. Herr, “Low-latency tracking of multiple permanent magnets,” IEEE Sensors Journal, vol. 19, no. 23,
pp. 11 458-11 468, 2019.
[72] S. Song, X. Qiu, J. Wang, and M. Q.-H. Meng, “Design and optimization strategy of sensor array layout for magnetic localization system,” IEEE Sensors Journal, vol. 17, no. 6, pp. 1849-1857, 2017.
[73] M.-C. Kim, E.-S. Kim, J.-O. Park, E. Choi, and C.-S. Kim, “Robotic localization based on planar cable robot and hall sensor array applied to magnetic capsule endoscope,” Sensors, vol. 20, no. 20, p. 5728, 2020.
[74] J. D. Jackson, “Classical electrodynamics,” 1999.
[75] J. Shawash and D. R. Selviah, “Real-time nonlinear parameter estimation using the levenberg-marquardt algorithm on field programmable gate arrays,” IEEE Transactions on industrial electronics, vol. 60, no. 1, pp. 170-176, 2012.
[76] J. Nocedal and S. J. Wright, Numerical optimization. Springer, 1999.
[77] J. Wu and L. Zhou, “Control co-design of actively controlled lightweight structures for high-acceleration precision motion systems,” in 2022 American Control Conference (ACC). IEEE, 2022, pp. 5320-5327.

1. An apparatus comprising:
a permanent magnet;
a segmented tubular member comprising a first end and a second end, wherein:
the permanent magnet is coupled to the first end of the segmented tubular member;
the second end of the segmented tubular member is configured to receive a distal end of a catheter; and
an electromagnet; and
a controller, wherein the controller is configured to:
control an electrical current to the electromagnet; and
control a magnetic force exerted by the electromagnet on the permanent magnet.

2. The apparatus of claim 1 wherein:
the permanent magnet is a first permanent magnet; and
the apparatus further comprises a second permanent magnet coupled to the segmented tubular member between the first end of the segmented tubular member and the second end of the segmented tubular member.

3. The apparatus of claim 2 further comprising a third permanent magnet coupled to the segmented tubular member between the first end of the segmented tubular member and the second end of the segmented tubular member.

4. The apparatus of any one of claims 1-3 further comprising a drive mechanism configured to engage a catheter inserted into the first end of the segmented tubular member.

5. The apparatus of any one of claims 1-4 further comprising a catheter inserted into the first end of the segmented tubular member.

6. The apparatus of any one of claims 1-5 wherein the permanent magnet comprises a central channel.

7. The apparatus of any one of claims 1-6 wherein the electromagnet comprises a pair of Helmholtz coils.

8. The apparatus of claim 7 wherein:
the pair of Helmholtz coils is a first pair of Helmholtz coils;
the electromagnet comprises a second pair of Helmholtz coils; and
the controller is configured to control a magnetic force exerted by the electromagnet on the permanent magnet in three-dimensional space.

9. The apparatus of claim 8 wherein the electromagnet comprises a third pair of Helmholtz coils.

10. The apparatus of any one of claims 1-7 further comprising an imaging device.

11. The apparatus of claim 10 wherein the imaging device is configured to provide imaging data of the permanent magnet.

12. The apparatus of any one of claims 1-11 wherein the permanent magnet and the segmented tubular member each have an outer diameter between 0.1 millimeters and 10 millimeters.

13. The apparatus of any one of claims 1-11 wherein the permanent magnet and the segmented tubular member each have an outer diameter less than or equal to 6.0 millimeters.

14. The apparatus of any one of claims 1-11 wherein the permanent magnet and the segmented tubular member each have an outer diameter less than or equal to 4.0 millimeters.

15. The apparatus of any one of claims 1-13 wherein the segmented tubular member comprises a rectangular pattern of voids.

16. The apparatus of any one of claims 1-15 further comprising a magnetic sensor configured to detect a position of the permanent magnet.

17. The apparatus of claim 16 wherein the magnetic sensor is configured as an array of sensors circumscribing the permanent magnet.

18. The apparatus of claim 16 or claim 17 wherein the sensor is coupled to the controller.

19. The apparatus of any one of claims 1-18 further comprising a visual display configured to display a position of the permanent magnet.

20. A method of directing a catheter into a lumen, the method comprising:
inserting the catheter into a segmented tubular member, wherein:
the segmented tubular member comprises a first end and a second end;
the segmented tubular member comprises a permanent magnet coupled to the first end; and
the catheter is inserted into the second end of the segmented tubular member;
advancing the catheter and the segmented tubular member into the lumen; and
exerting a magnetic force on the permanent magnet.

21. The method of claim 20 wherein the lumen is an artery with an inner diameter greater than or equal to 6.0 millimeters.

22. The method of any one of claims 20-21 wherein the magnetic force is generated by an electromagnet.

23. The method of claim 22 further comprising varying the magnetic force generated by the electromagnet by controlling an electrical current to the electromagnet.

24. The method of any one of claims 22-23 wherein the electromagnet comprises a pair of Helmholtz coils.

25. The method of any one of claims 20-24 wherein advancing the catheter and the segmented tubular member into the lumen comprises engaging the catheter with a drive mechanism.

26. The method of any one of claims 20-25 wherein:
the permanent magnet comprises a central channel; and
the method further comprises inserting an instrument through the central channel.

27. The method of any one of claims 20-26 further comprising obtaining imaging data of the permanent magnet.

28. The method of any one of claims 20-27 wherein the permanent magnet and the segmented tubular member each have an outer diameter less than or equal to 6.0 millimeters.

29. The method of any one of claims 20-28 wherein the permanent magnet and the segmented tubular member each have an outer diameter less than or equal to 4.0 millimeters.

30. The method of any one of claims 20-29 wherein the segmented tubular member comprises a rectangular pattern of voids.

31. A method of making a segmented tubular member, the method comprising:
obtaining a flexible tubular member;
obtaining a pattern;
flattening the flexible tubular member;
positioning the pattern on the flexible tubular member;
using a cutting instrument to remove material from flexible tubular member, wherein the cutting instrument follows the pattern; and
removing the pattern from the flexible tubular member.

32. A method of directing a magnetic element into a lumen, the method comprising:
obtaining a radiographic image of the lumen, wherein:
the lumen comprises a non-bifurcated portion and a bifurcated portion; and
the bifurcated portion of the lumen comprises a target branch and a non-target branch;
inserting the magnetic element into the non-bifurcated portion of the lumen, wherein a position of the magnetic element is tracked via a magnetic sensor until the magnetic element is proximal to the bifurcated portion;
imaging the magnetic element via radiography;
directing the magnetic element into the target branch of the bifurcated portion of the lumen via a magnetic force on the permanent magnet;
advancing the magnetic element into the target branch of the bifurcated portion of the lumen;
ceasing imaging of the magnetic element via radiography; and
detecting the position of the magnetic element via a magnetic sensor.

33. The method of claim 32 wherein the magnetic element is coupled to a distal end of a catheter.

34. The method of claim 32 or claim 33 wherein the lumen is an artery.

35. The method of any one of claims 32-34 wherein imaging the magnetic element via radiography comprises x-ray imaging.

36. The method of any one of claims 32-35 wherein the magnetic force on the permanent magnet is generated by an electromagnet.

37. The method of any one of claims 32-35 wherein the magnetic force on the permanent magnet is generated by a pair of Helmholtz coils.

38. The method of any one of claims 33-37 wherein: advancing the magnetic element into the target branch of the bifurcated portion of the lumen comprises engaging the catheter with a drive mechanism.

39. The method of any one of claims 32-38 wherein:
the permanent magnet comprises a central channel; and
the method further comprises inserting an instrument through the central channel.

40. The method of any one of claims 32-39 wherein the permanent magnet has an outer diameter less than or equal to 6.0 millimeters.

41. The method of any one of claims 32-39 wherein the permanent magnet has an outer diameter less than or equal to 4.0 millimeters.

42. The method of any one of claims 33-41 further comprising inserting the distal end of the catheter into a segmented tubular member prior to inserting the magnetic element into the non-bifurcated portion of the lumen.

43. The method of claim 42 wherein the segmented tubular member comprises a rectangular pattern of voids.

44. An apparatus comprising:
a permanent magnet;
an electromagnet;
a magnetic sensor configured to detect a position of the permanent magnet; and
a controller, wherein the controller is configured to:
control an electrical current to the electromagnet; and
control a magnetic force exerted by the electromagnet on the permanent magnet.

45. The apparatus of claim 44 further comprising a segmented tubular member, wherein:
the segmented tubular member comprises a first end and a second end; and
the permanent magnet is coupled to the first end of the segmented tubular member.

46. The apparatus of claim 45 wherein the second end of the segmented tubular member is configured to receive a distal end of a catheter.

47. The method of any one of claims 44-46 wherein the electromagnet comprises a pair of Helmholtz coils.

48. The apparatus of claim 47 wherein:
the pair of Helmholtz coils is a first pair of Helmholtz coils;
the electromagnet comprises a second pair of Helmholtz coils; and
the controller is configured to control a magnetic force exerted by the electromagnet on the permanent magnet in three-dimensional space.

49. The apparatus of claim 48 wherein the electromagnet comprises a third pair of Helmholtz coils.

50. The apparatus of any one of claims 44-49 further comprising an imaging device.

51. The apparatus of claim 50 wherein the imaging device is configured to provide imaging data of the permanent magnet.

52. The apparatus of any one of claims 45-51 wherein the permanent magnet and the segmented tubular member each have an outer diameter between 0.1 millimeters and 10 millimeters.

53. The apparatus of any one of claims 45-52 wherein the permanent magnet and the segmented tubular member each have an outer diameter less than or equal to 6.0 millimeters.

54. The apparatus of any one of claims 45-53 wherein the permanent magnet and the segmented tubular member each have an outer diameter less than or equal to 4.0 millimeters.

55. The apparatus of any one of claims 45-54 wherein the segmented tubular member comprises a rectangular pattern of voids.

56. A method of detecting a position of a magnetic element, the method comprising:
(a) placing the magnetic element in a plurality of known positions;
(b) estimating a position of the magnetic element by measuring a magnetic field of the magnetic element in the plurality of known positions via a plurality of magnetic sensors; and
(c) calculating a position of the magnetic element, wherein:
each magnetic sensor in the plurality of magnetic sensors is located in a fixed position; and
calculating the position of the magnetic element is performed via a nested least square method.

57. The method of claim 56 wherein the nested least square method minimizes discrepancies between the known position of the magnetic element and the estimated position of the magnetic element using the measured magnetic field and the fixed position of the sensor.
